(19) United States
(12) Patent Application Publication
(10) Pub. No.: US 2020/0345971 A1
(43) Pub. Date: Nov. 5, 2020

(54) WEARABLE THERMAL DEVICES AND METHODS OF USING THEM

(71) Applicant: Ebb Therapeutics, Inc., Pittsburgh, PA (US)

(72) Inventors: Jeffrey J. SCHIRM, Monroeville, PA (US); Robert E. TUCKER, Sanibel, FL (US); Rochak CHADHA, Pittsburgh, PA (US); Jeremy P. GRODKIEWICZ, Portland, OR (US); Aislinn T. TAN, Portland, OR (US)

(21) Appl. No.: 16/863,978

(22) Filed: Apr. 30, 2020

Related U.S. Application Data
(60) Provisional application No. 62/841,150, filed on Apr. 30, 2019.

(51) Int. Cl. A61M 21/02; A61F 7/02

(52) U.S. Cl. CPC A61M 21/02; A61F 2007/0007; A61F 2007/0212; A61F 7/02

(57) ABSTRACT

Described herein are forehead-mounted temperature regulation apparatuses and methods of using them. These apparatuses may be used for improving sleep. Also described herein are methods of improving sleep using these apparatuses.

### CROSS REFERENCE TO RELATED APPLICATIONS

This patent application claims priority to U.S. Provisional Patent Application No. 62/841,150, titled “WEARABLE THERMAL DEVICES AND METHODS OF USING THEM,” filed on Apr. 30, 2019, and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The apparatuses and methods described herein may be used to improve sleep, including reducing sleep onset, improving sleep maintenance, increasing sleep duration, reduce awakenings, and increasing deep sleep relative to light sleep in a subject, as well as to promote relaxation, improve mood, and/or treat one or more neurological disorders.

### BACKGROUND

Sufficient and high-quality sleep is important for an individual's physical, mental, and emotional health. Despite various drugs and devices on the market for enhancing sleep and treating sleep disorders, disruption and irregularities, including insomnia, resulting in poor sleep is a widespread and pervasive problem. For example, previously described devices and techniques for the treatment of sleep disorders have included the use of cooling therapies, including applying cooling therapy to a subject's forehead, to enhance sleep. There is evidence for enhancing sleep by cooling a subject's skin (e.g., forehead), perhaps by taking advantage of a mechanism involving cooling an underlying brain region. Recent work by suggests that warming may also result in sleep enhancement, particularly warming relative to ambient temperature. The mechanism of action that temperature has on sleep has not been conclusively identified.

Body temperature regulation (thermoregulation) is a fundamental homeostatic function that is regulated by the central nervous system. The preoptic area (or POA) of the hypothalamus is considered the most important thermoregulatory site in the brain on the basis of thermoregulatory studies, such as responses elicited by local warming and cooling, analysis of lesions, results from stimulation and single neuronal recording, and other techniques. Thermoregulation in the preoptic area is controlled by thermosensitive neurons. The thermosensitive neurons in the POA receive and integrate cutaneous (skin) and deep body thermal information. These neurons are tonically active at thermoneutral temperature, and control the thermoregulatory efferent pathway.

A thermoreceptor may be a peripheral thermoreceptor (e.g., a receptor on the skin or mucous membranes of a subject that monitors external temperature) or a central thermoreceptor (e.g., an internal receptor that monitors internal body temperature). The preoptic area of the hypothalamus contains temperature sensitive neurons (warm sensitive neurons (WSN) and cold sensitive neurons (CSN)). These neurons were identified in the preoptic area on the basis of in vivo and in vitro studies. Warm sensitive neurons are directly sensitive to (and fire in response to) locally warm temperatures and cold sensitive neurons are directly sensitive to (and fire in response to) local cool temperatures.

Among body regions, the forehead has unique physiological and neuroanatomical properties that suggest it may play a prominent role in influencing the thermoregulatory hypothalamic modulation. The distribution of warm and cold spots has been shown to be highest over the face and forehead of all body parts Thermal sensation has been shown to be highest in the forehead of all body parts. Further, the forehead comprising glabrous (non-hairy) skin has been shown to play a prominent role in the body response to thermoregulation given that the heat transfer function and efficacy of glabrous skin is unique within the entire body based on the capacity for a very high rate of blood perfusion and the novel capability for dynamic regulation of blood flow.

It would be very useful to provide systems and methods for evoking a response in a subject's body to improve sleep, relaxation and/or mental states (e.g., mood) in the subject.

### SUMMARY OF THE DISCLOSURE

Described herein are methods and apparatuses capable of stimulating a response in a subject's body to improve sleep, relaxation and/or mental states (e.g., mood) in the subject and/or to treat one or more neurological disorders. These methods, systems and devices may stimulate temperature sensitive receptors (e.g., cold sensitive or warm sensitive neurons) in a subject's skin, and in particular in the subject's face, including the forehead. In particular, the apparatuses and methods described herein may use a powered (e.g., battery and/or wall powered), wearable device (e.g., wearable on a forehead) apparatus that may be useful to treat a subject. For example, these devices may be useful to treat sleep, modulate mood and/or increase relaxation. For example, these devices may be useful to decrease sleep onset (e.g., facilitate falling to sleep), decrease arousals, increase sleep duration, increase depth of sleep, and/or treat insomnia).

The apparatuses and systems described herein are particularly well suited for comfort and to avoid irritation. For example, these apparatuses may be adapted to reduce or eliminate the noise and vibration of operation. These apparatuses may also direct or redirect the airflow used to cool the thermoelectric components in a manner that aids in the therapeutic effect. The thermoelectric components may include one or more thermoelectric (TEC) devices which may also be referred to here as thermoelectric temperature regulators (TERs).

In some variations, the apparatuses and methods described herein, including methods of using the apparatuses, may be used to enhance sleep. Enhancing sleep may include one or more of: reducing sleep onset latency, extending sleep duration, reducing awakenings, and/or increasing the duration of deeper sleep stages relative to stage 1 sleep in a subject (e.g., increasing the ratio of deeper sleep stage duration relative to stage 1 sleep duration). These apparatuses and methods may be applied to subjects (e.g., persons, patients, etc.) in need thereof; for example, the methods described herein may be used to treat a subject suffering from a sleeping disorder such as insomnia. In general, these apparatuses and methods apply (and may maintain) stimulation of temperature-sensitive receptors in a subject's body (skin) and through the receptor provide signals that enhances the subject's sleep. Any of the apparatuses and methods of using them described herein may be configured to apply heat and/or cooling to a subject's forehead using a self-contained apparatus with or without a hand-held control.

For example, described herein are apparatuses that may include: a conformable body configured to be worn on the subject's forehead having one or more thermally conductive skin-facing surfaces; one or more thermoelectric temperature regulators in thermal communication with the one or more skin-facing surfaces; one or more cooling fans coupled to the conformable body through a mechanical damping member that is configured to dampen vibrations (e.g., greater than 25 Hz, greater than 30 Hz, greater than 50 Hz, greater than 60 Hz, between about 25-800 Hz, between about 30-750 Hz, between about 50 and 700 Hz, between about 100 and 800 Hz, between about 150 and 800 Hz, between about 200 and 800 Hz, between about 200 and 700 Hz, between about 250 and 600 Hz, etc.); and a control unit comprising a controller configured to apply power to the one or more thermoelectric temperature regulators to cool or warm the one or more thermally conductive skin-facing surfaces to between 1° C.-30° C.

For example, an apparatus may include: a conformable body configured to be worn on the subject's forehead having one or more thermally conductive skin-facing surfaces; one or more thermoelectric temperature regulators (TERs) in thermal communication with the one or more skin-facing surfaces; one or more cooling fans coupled to the conformable body through a mechanical damping member; and a controller configured to apply power to the one or more TERs to cool or heat the one or more thermally conductive skin-facing surfaces to between 1° C.-30° C.

The conformable body may refer to the ability of the skin-facing surfaces, which may be part of a series of rigid portions connected by flexible regions, to conform to the curved surface of a variety of different user head (forehead) shapes. In some variations this may be achieved by connecting the rigid portions (which may each include a TEC/TER and a fan to cool the TEC/TER) with a flexible hinging region. For example, the conformable body may include a plurality of rigid sections arranged in a long axis and linked by flexible hinge regions that are adapted to bend in a hinge direction perpendicular to the long axis and to permit rotation of a least 5 degrees (e.g., +/−3 degrees, +/−5 degrees, +/−7 degrees, +/−10 degrees, +/−12 degrees, +/−15 degrees, etc.) in the long axis. The long axis may be curved or curving and generally conforms to the curvature of the head (e.g., forehead).

The mechanical damping member may be any appropriate mechanical damping material. The mechanical damping member may have a loss factor of greater than 0.8 (e.g., 0.85 or greater, 0.90 or greater, 0.95 or greater, 1 or greater, etc.). Damping is the dissipation of energy, usually by releasing it in the form of low-grade heat. The loss factor (η) is the ratio of energy dissipated from the system to the energy stored in the system for every oscillation. It is often useful to relate the loss factor to the damping ratio. The damping ratio can be approximated from the loss factor. A loss factor of 0.1 is generally considered a minimum value for significant damping.

In some variations the mechanical damping member may be configured to dampen vibrations between a range of about 25 to 6000 Hz (e.g., between about 25-800 Hz, between about 50-5000 Hz, between about 25-1000 Hz, between about 50-2000 Hz, between about 200-3000 Hz, between about 300-6000 Hz, between about 1000-2000 Hz, between about 2000-4000 Hz, between about 4000-5000 Hz, between about 5000-6000 Hz, between about 4000-6000 Hz, between about 3000-6000 Hz, between about 100 Hz-900 Hz, between about 200 Hz to 7000 Hz, between about 300 and 600 Hz, etc.). The mechanical damping member may be configured as a sheet or layer, a grommet, a bushing, a washer, a sleeve, a pad, etc. The mechanical damping member(s) may allow the fan to be vibrationally isolated from the other portions of the apparatus, including in particular a cover or housing over the fan, the conformable body, etc.

In some variations, the mechanical damping member may comprise a foam; this form may be an open-cell foam. For example, in some variations, the mechanical damping member may include a seat in which the fan (and/or other portions of the fan subassembly) may sit without any rigid connection to the wearable body of the apparatus, e.g., the conformable body. This may prevent bone conduction of vibration of the fan, which may be oscillating at, e.g., between 20-300 Hz; the damping member may be coordinated with the oscillation rate of the fan, to prevent conduction of the fan vibration.

Other examples of materials that may be used as a mechanical dampening member may include (but are not limited to) thermoplastic material, including thermoplastic vulcanizates (e.g., ISODAMP 6000). As mentioned, these materials may be used to vibrationally isolate the fans from all or parts of the apparatus. Vibrational isolation as used herein does not require perfect isolation, but may be passive vibrational isolation that dampens a substantial amount of the vibration due to the fan, particularly at the frequency of oscillation of the fan (e.g., between about 10,000 rpm and 40,000 rpm).

The cooling fans may be enclosed in a housing and may be separated from the housing by the mechanical damping member(s). The housing may include vents (e.g., fins, channels, etc.) to direct the airflow from off of the apparatus, driven by the fan(s), in a particular direction (e.g., parallel to the skin surface when the device is worn and/or away from the face, or toward the top of the head), as described in more detail below.

In any of the apparatuses described herein the conformable body may extend in a long axis (which may be straight or curved), and the one or more thermally conductive skin-facing surfaces may be divided into a plurality of thermally conductive skin-facing surfaces that are pivotally coupled to each other so as to permit each thermally conductive skin surface to rotate in the long axis relative to each other. The individual surface may be flat or curved (e.g., concave).

For example, the conformable body may comprise a plurality of rigid sections arranged in a long axis and linked by flexible hinge regions that are adapted to bend in a hinge direction perpendicular to the long axis and to permit rotation of a least x degrees in the long axis (where x may be, e.g., +/−3 degrees, +/−5 degrees, +/−7.5 degrees, +/−8 degrees, +/−9 degrees, +/−10 degrees, +/−12 degrees, +/−15 degrees, +/−, etc.). The flexible hinge region may be formed of a flexible material, such as a fabric. The rotation in the long axis may be limited (e.g., to less than +/−20 degrees, +/−18 degrees, +/−15 degrees, +/−12.5 degrees, +/−, 10 degrees, +/−9 degrees, +/−8 degrees, +/−7 degrees, etc.). The rotation in the long axis may refer to the relative rotation between the adjacent more rigid regions. The rotation in the long axis may in part be due to one a cut-in region at the hinge between each ridged region.

In any of these apparatus, the controller may be configured to apply a power control scheme to the one or more thermoelectric temperature regulators to regulate the temperature of the one or more thermally conductive skin-facing surfaces. For example, the controller may be configured to a establish a set temperature of between 1° C.-30° C., wherein the power control scheme may vary the power so that the temperature of the one or more thermally conductive skin-facing surfaces deviates from the set temperature by between 1-5 degrees C.

In some variations, the controller may be configured to apply a power control scheme to the one or more TERs comprising: applying power to the one or more TERs to cool or heat the one or more thermally conductive skin-facing surfaces to a set temperature of between 1-30° C., holding the set temperature for between 1 and 15 seconds, reducing or stopping power to the one or more TERs until a temperature of the thermally conductive skin-facing surface varies from the set temperature by a relax temperature of between 1-5° C., and repeating these steps.

Any of the controllers described herein may adjust the power control scheme in a predetermined (or pre-set) manner or in an adaptive manner, e.g., based on one or more inputs. For example, in some variations, the power control scheme may be configured to dynamically change the dwell time. For example, the power control scheme may be configured to decrease (or increase) the dwell time as the apparatus is continuously operated. In some variations, the increase may be non-linear; for example, the dwell time may be decreased (e.g., by about 10%, 20%, 30%, 40%, 50%, 60%, 70%, 80%, 90%, 100%, 120%, etc.) then allowed to remain the same for a period of time before again increase the decreased. In some variations the dwell time may be changed by in a random or pseudo-random manner.

Alternatively or additionally, the controller may modify the deviation of temperature that the thermally conductive skin-facing surface is allowed to reach to once the controller reduces or (more preferably) stops the power to the thermoelectric temperature regulator (e.g., between 1-5° C.). This temperature may be referred to as the relax temperature, and may be set (e.g., to about 1 degree, about 2 degrees, about 3 degrees, about 4 degrees, about 5 degrees, etc.) or it may be dynamically adjusted by the controller. In some variation the relax temperature may be increased as the device is continuously operated. In some variations the relax temperature is varied randomly or pseudo-randomly. Although the majority of the examples described herein include a relax temperature of between 1-5 degrees C., in some variations this relax temperature may be between 1-10 degrees C. (e.g., between 1-9 degrees C., between 1-8 degrees C., between 1-7 degrees C., between 1-6 degrees C., etc.). In some variations the relax temperature is between 2-10 degrees C., between 3-10 degrees C., between 4-10 degrees C., between 5-10 degrees C., between 2-8 degrees C., between 2-9 degrees C., etc.

Adjusting either or both the dwell time and the relax temperature may prevent desensitization of the user's forehead to the decreased temperature and/or may help save power in the device (e.g., battery power in variations including a battery). In particular, in variations in which the controller shuts off power to the thermoelectric temperature regulators during this portion of the power control scheme.

In any of the methods and apparatuses described herein the temperature of the thermally conductive skin-facing surface may be directly measured by one or more sensors (e.g., thermistors, IR sensors, thermocouple, resistance temperature detector, etc.), e.g., in contact with the thermally conductive skin-facing surface, or may be in contact with a thermally conductive component that is in thermal contact with the thermally conductive skin-facing surface, such as the thermoelectric temperature regulator(s). In some variations each thermoelectric temperature regulator includes one or more temperature sensors on a portion of the thermoelectric temperature regulator in thermal contact with the thermally conductive skin-facing surface.

In general the controllers described herein may control the power to the thermoelectric temperature regulators as well as to the cooling fans. In some variations the cooling fans may separately controlled. For example, the cooling fans may be powered continuously and/or constantly. In some variations, the cooling fans may be turned on/off by the controller, and/or the power (and/or the rotational speed) may be adjusted by the controller based on the temperature of the thermoelectric temperature regulator or a power control scheme.

For example, the controller may be configured to apply energy in a pulsatile manner to either or both the cooling fans and the thermoelectric temperature regulators.

Any of these apparatuses may include a removable comfort layer (comfort layer assembly) that may be removably attached to the conformable body over the one or more thermally conductive surfaces. The comfort layer may include a plurality of windows of thermally conductive material configured to interface over the thermally-conductive skin-facing surfaces. These windows may be any appropriate thermally-conductive material, including but not limited to polyurethane.

Any of these apparatuses may include a removable cover assembly configured to attach over the conformable body to cover the one or more cooling fans. The cover assembly may include a plurality of vents (e.g., openings, channels, etc.) to allow airflow there through. As described herein, the airflow may be directed, e.g., away from the subject's face (e.g., their nose, mouth, etc.) and towards the top of their head or forehead (e.g., hairline). In some variations the airflow may be directed away from the subject's skin. In some variations, the airflow may be directed across the subject's skin.

Any of these apparatuses may include a holdfast configured to secure the conformable body to the subject's forehead. For example, the holdfast may comprise a headband, such as a headband having a plurality of interlaced straps configured to be stretched and held in place to maintain tension on the conformable body to ensure contact with the skin. The holdfast may be a strap.

The control unit may be enclosed in a housing connected by an elongate flexible cable (e.g., cord, wire, etc.) to the one or more thermoelectric temperature regulators. The apparatus may also include a battery in the housing. The housing may be sized to be held in subject's hand (e.g., “handheld”) and/or may be configured to be set on a table, bed or nightstand when the device is worn, or may be configured to attach to the subject's body or clothing. The housing may include circuitry, including the controller circuitry, a clock circuitry, etc.

Any of these apparatuses may include one or more sensors for sensing user activity or state. As mentioned, any of these apparatuses may be configured to detect the temperature of the skin-facing surface and/or of the subject wearing the device. In some variations, the apparatus includes one or more sensors configured to detect one or more of the subject's autonomic state and the subject's sleep/wake state. Sensors may include, but are not limited to: motion sensors (e.g., accelerometers), electric skin conductance sensors, heart rate sensors, electro-ocular sensors, etc.

Any of these apparatuses may include a wireless communications circuit configured to wirelessly transmit and receive to and from the controller.

In some variations, an apparatus includes: a conformable body configured to be worn on a subject's forehead and having a plurality of rigid sections arranged in a long axis and linked by flexible hinge regions that are adapted to bend along the long axis and to permit rotation of a least 5 degrees in the long axis; a thermoelectric temperature regulator on each rigid section; a thermally conductive skin-facing surface on each rigid section, wherein the thermally conductive skin-facing surface is in thermal communication with the thermoelectric temperature regulator on each rigid section; a cooling fan on each rigid section, configured to cool the thermoelectric temperature regulator on the same rigid section; and a controller configured to apply power to all of the thermoelectric temperature regulators to cool or warm the thermally conductive skin-facing surfaces on each rigid section to between 1° C.-30° C.

For example, an apparatus may include: a conformable body extending in a long axis and configured to be worn on the subject's forehead having a plurality of thermally conductive skin-facing surfaces, wherein each thermally conductive skin-facing surface is coupled to an adjacent thermally conductive skin-facing surface through a pivoting connection that permits each thermally conductive skin surface to rotate in the long axis relative to the adjacent skin-facing surface; a plurality of thermoelectric temperature regulators, wherein each thermally conductive skin-facing surface is in thermal communication with one of the thermoelectric temperature regulators of the plurality of thermoelectric temperature regulators; a plurality of cooling fans, wherein each thermoelectric temperature regulators of the plurality of thermoelectric temperature regulators is cooled by a fan of the plurality of cooling fans; and a control unit comprising a controller configured to apply power to the plurality of thermoelectric temperature regulators to cool the thermally conductive skin-facing surface to between 1° C.-30° C. Each fan may be coupled to the conformable body through a mechanical damping member, as mentioned above. The fan may therefore “float” relative to the subject-contacting body of the device, or may otherwise be vibrationally isolated, to prevent conduction of vibrations from the fan or fan assembly.

For example, an apparatus may include: a conformable body configured to be worn on the subject's forehead having one or more thermally conductive skin-facing surfaces; one or more thermoelectric temperature regulators in thermal communication with the one or more skin-facing surfaces; one or more cooling fans coupled to the conformable body through a mechanical damping member; and a control unit comprising a controller configured to apply a power controls scheme to the one or more thermoelectric temperature regulators. For example, the controller may be configured to apply a power control scheme to the thermoelectric temperature regulators comprising: (a) applying power to the thermoelectric temperature regulators to cool or heat the thermally conductive skin-facing surface(s) in thermal communication with the thermoelectric temperature regulators to a set temperature of between 1-30° C., (b) holding the set temperature for between 1 and 15 seconds, (c) reducing or stopping power to the thermoelectric temperature regulator until a temperature of the thermally conductive skin-facing surface varies from the set temperature by a relax temperature of between 1-5° C., and repeating steps (a)-(c).

As mentioned above, the apparatuses described herein may generally include a fan of fan subassembly for regulating the temperature of the thermoelectric regulator. For example, the fan subassembly may be configured as a thermoelectric regulator assembly and may include heat sinks, cooling fans and thermally conductive interface materials. For example, in some variations, the cooling fans may be configured to be isolated within foam to reduce vibration transmission.

Any of these devices described herein may include a conformable body that may be configured to be worn on a subject's forehead. The conformable body may be configured to ensure that each thermoelectric regulator assembly (in particular, the thermal transfer skin-facing surface) is held in contact with the subject's skin without applying too much (or too little) force. This contact may be direct or indirect (e.g., through a comfort layer. As mentioned above, each skin-facing surface may be coupled to an adjacent thermally conductive skin-facing surface through a pivoting connection that permits each thermally conductive skin surface to rotate in the long axis relative to the adjacent skin-facing surface. The hinged and/or pivoting connection between the skin-facing surfaces (e.g., between each thermoelectric regulator assembly) may be configured as a pivoting joint, which may be formed of a flexible material that is notched or forms a narrowing waist, permitting bending and twisting. The conformable body may therefore be configured to allow each thermoelectric assembly to independently move in different directions (e.g., out of a plat plane) to conform to the shape of a subject's forehead.

In general, the apparatuses and methods of using them described herein may optimize skin contact between the thermal transfer region(s) of the apparatus and the subject. In some variations the pivoting connection may be a ball-and-socket joint, a gimbal, etc. The pivoting connection typically allows the thermal transfer regions (e.g., the thermoelectric regulator assembly regions) to move in two axis, e.g., to bend or twist in both the perimeter of the head and the direction perpendicular to that.

A holdfast may be used with or included as part of the apparatuses described herein. The holdfast may be configured to apply forcer to ensure a quality skin contact between the thermal transfer regions of the skin-facing surfaces of the thermoelectric regulator assembly and the subject's skin. The pivoting connections described herein may be configured to allow the distribution of force between the skin-facing surfaces.

Any of the thermal transfer regions of the skin-facing surface of the thermoelectric regulator assembly may include a thermal transfer material, such as a gel pad (e.g., hydrogel), that may be open or enclosed. A gel pad may be thin, and may soften the edges of the thermal transfer region. In some variations the thermal transfer region may be formed of a thermally conductive metal, such as copper, stainless steel, aluminum, etc. In general, the better the contact between the thermal transfer region and the patient's skin, the more responsive the patient's skin may be to even smaller changes in temperature. In general, the thermal transfer region(s) of the apparatus may be in full surface contact with the TEC interface material.

As already discussed above, the fan (e.g., fan sub-assembly) may be enclosed or at least partially enclosed to hold the fan in vibrational isolation relative to the rest of the apparatus. For example, the fan may be cushioned in a foam material that acts to dampen any vibration due to operation of the fan. The fan sub-assembly may also include one or more heat sinks which may include one or more openings in the fan enclosure to direct the cooling airflow from the fan up over the top of head and/or down over the lower face. Such openings may preventing airflow from being directed towards any adjacent thermoelectric regulator assemblies.

The replaceable comfort layer (comfort layer assembly) may be attached to the conformable body and held in place between the thermoelectric regulator and the skin surface. The comfort layer may be formed of one or more integral layers of materials such as foams and fabrics that may be configured at least partially around the thermal electric regulator surface, e.g., between the thermal transfer region and the skin of the patient, and may be contoured to help ensure optimal thermoelectric regulator skin contact while providing a soft and comfortable interface to the forehead area minimizing pressure contact points. Any of the comfort layers described herein may further comprising one or more windows of a material such as polyurethane to provide an interface between the thermoelectric regulator assembly and the skin.

As mentioned, any of these apparatuses may also include a cover assembly that may be attached to the conformable body to cover the side of the apparatus (e.g., the outside of the thermoelectric regulator assembly), the region facing away from the skin surface. A cover assembly can be made of porous fabrics that allow adequate airflow through the fabric for the cooling fans and exhaust airflow while providing an adequate barrier to prevent hair from contacting the fan. The cover assembly may be removable for cleaning.

Any of the holdfasts described herein may include a user-replaceable hold down or headband to maintain contact of the conformable body assembly to the forehead. For example, a headband may be configured with two or more interlaced straps that can be stretched and held in place (e.g., with a latch-and-hook material, such as Velcro) to maintain the appropriate tension on the conformable body to ensure thermoelectric regulator assembly contact with the skin.

An apparatus as described herein may include: a conformable body configured to be worn on the subject's forehead having one or more thermally conductive skin-facing surfaces; a plurality of thermoelectric temperature regulators in thermal communication with the one or more skin-facing surfaces; a plurality of cooling fans coupled to the conformable body through a mechanical damping member; and a controller configured to apply a power controls scheme to the thermoelectric temperature regulators to cool the one or more thermally conductive skin-facing surfaces by repeatedly: applying power to the thermoelectric temperature regulator to cool or heat the thermally conductive skin-facing surface in thermal communication with the thermoelectric temperature regulator to a set temperature of between 1-30° C., maintaining the set temperature for a dwell time of between 1 and 15 seconds, and reducing or stopping power to the thermoelectric temperature regulator until a temperature of the thermally conductive skin-facing surface varies from the set temperature by a relax temperature of between 1-5° C.

In general, the controller controlling the thermoelectric regulators described herein may provide either a predetermined or a subject selectable power scheme to the thermoelectric regulators. A predetermined power scheme may apply constant power for a predetermined or subject-selectable temperature set point and/or may automatically vary the dwell time and/or relax temperature (e.g., the temperature to which the TEC/TERs are allowed to vary to after turning the power off or down), or could apply a predetermined variable power application to vary the temperature profile to a predetermined or subject selectable temperature set point. Such a variable power application may ramp the cooling temperature to a target temperature over a period of time, may hold the temperature for a period of time and allow the temperature to raise or lower to a second set point for another period of time. The waveform of an applied power scheme may be any appropriate predetermined shape or combination of shapes, such a square waveform, saw tooth waveform, a sinusoidal or other custom waveform patterns. Any of these waveforms may have variable amplitudes and frequencies. Alternatively, a control (e.g., user control) on or in communication with the apparatus may allow the subject to manipulate one or more (e.g., all) of the parameters of power scheme profile or to select between profiles.

In general, the controller maybe integrated with the conformable body or may be attached to a stand-alone controller, e.g., wirelessly or via a flexible wire. Thus, in some variations, the controller may communicate to the regulators through a wireless connection. As will be described in more detail below, the controller be connected to the rest of the device through wire connected, e.g., to a hand-held housing. Thus, a flexible wire may be can be routed from the conformable body in a desirable direction by the use of a routing channel, guide (e.g., loop, etc.) that attaches the flexible wire to the headband. A routing channel could include self-securing (e.g., Velcro material) strap that may attach to the flexible wire and the headband.

For example, described herein are apparatuses for applying thermal energy to a subject's forehead comprising: a conformable body configured to be worn on the subject's forehead having a skin-facing surface; a plurality of thermoelectric temperature regulators arranged across the skin-facing surface; a controller configured to apply power to cool the thermoelectric temperature regulators to between 1° C.-30° C.; and a holdfast configured to secure the apparatus to the subject's forehead. The holdfast may be a strap, hat, headband, adhesive, or the like. Any of the apparatus features described above may be included in this variation.

For example, an apparatus as described herein may include: a conformable body configured to be worn on the subject's forehead having a skin-facing surface; one or more thermoelectric temperature regulators arranged across the skin-facing surface; a thermal and vibration-damping material laterally arranged around the one or more thermoelectric temperature regulators; a control unit comprising a controller configured to apply power using a control scheme to cool the thermoelectric temperature regulators to between 1° C.-30° C.; and a holdfast configured to secure the conformable body to the subject's forehead. In any of these apparatuses, the control scheme may include any appropriate waveforms, which may be fixed or subject selectable.

The apparatus may include a plurality of cooling channels (e.g., fins) in communication with the one or more thermoelectric temperature regulators configured to direct airflow from the one or more thermoelectric temperature regulators and perpendicular to the skin-facing surface. In some variations the cooling channels may be configured to direct the airflow up and/or laterally when worn on the subject's head (e.g., away from the face). Alternatively, in some variations the cooling channels are configured to direct the airflow down (e.g., towards/over the subject's skin. The warmer air applied to other portions of the subject's head may allow the device to achieve a more profound effect, as the relative difference in temperature between the cooled (e.g., forehead) region and other parts of the body (e.g., laterally to the subject's ears, and/or up to the subject's crown region, and/or down to the subject's face) may enhance the physiological response.

Any of the apparatuses described herein may include a vibration-damping material between the thermoelectric temperature regulator and the skin-contacting surface(s). The vibration-damping material may be any appropriate material having sufficient properties, including vibrational damping, as described above. In some variations, the vibration-damping material may be foamed and/or porous; porosity may improve both the vibration-damping and ability to pass air through the material. The porosity may be greater than about 20 μm, about 50 μm, about 100 μm, about 150 μm, about 200 μm, about 250 μm, about 300 μm, about 400 μm, about 500 μm, about 600 μm, about 700 μm, about 800 μm, about 900 μm, about 1 mm, etc. The pores may be open cell or closed cell (or mixture thereof). For example, the vibration-damping material may comprise a foam.

In any of the methods and apparatuses described herein the skin-facing surface may comprise a dry material, and/or may be kept dry during use. The methods and apparatuses described herein do not require the use of a fluid or gel medium to assist in transferring of the temperature. However, in some variations, a thermally conductive gel material (e.g., formed of thermally conductive silicone) may be arranged as a layer or pad between the skin and the TEC. Alternatively or additionally, the apparatus may include materials like foams around the TECs to add to the conformability of the unit on the forehead.

In any of these methods and apparatuses, the control unit may be enclosed in a housing (e.g., a controller housing) connected by an elongate flexible wire to the conformable (e.g., wearable) body including the one or more thermoelectric temperature regulators and the skin-facing surface. The control unit may include one or more controls for operating the device (e.g., switches, knobs, touchscreens, etc.). The control unit may include an output, such as a display, dial, indicator, etc. In the some variations the housing may be configured as a hand-held housing. Thus, in some variations, the controller may be held in the users hand and use to turn the device on and off, and/or adjust the temperature, the on-time, the standby interval, etc. In some variations, the housing may enclose the power control circuitry and/or the power source (e.g., battery). In general, the controller may be contained in the housing. Thus, the apparatus may include a battery in the hand-held housing. Any of the apparatuses described herein may include one or more sensors configured to detect one or more parameters from the subject and/or the environment of the apparatus and/or the operation of the apparatus. For example, one or more temperature sensor (e.g., thermistor, etc.), accelerometer, IR sensor, etc. may be included. In some variations the apparatus may include one or more sensors to detect the subject's autonomic state and the subject's sleep/wake state. The sensor(s) may be present on the body (e.g., conformable body) and/or on the housing.

The apparatuses described herein may include one or more wireless communications circuits configured to wirelessly transmit and receive to and from the controller. The communications circuitry may be in the housing and/or the body of the apparatus. In some variations the controller is configured to apply energy in a pulsatile manner so that the thermoelectric temperature regulators apply pulsed cooling to the forehead. For example, the energy may be configured to be applied in pulses having a pulse duration of less than 360 seconds.

Any of these apparatuses may include a headband configured to hold the device onto the subject's forehead.

For example, an apparatus as described herein may include: a conformable body configured to be worn on the subject's forehead having a skin-facing surface; one or more thermoelectric temperature regulators arranged across the skin-facing surface; a foam vibration-damping material arranged at least partially around the one or more thermoelectric temperature regulators; a flexible cord extending from the one or more thermoelectric temperature regulators to a housing enclosing a control unit, the control unit comprising a controller configured to apply power using a control scheme to cool the thermoelectric temperature regulators to between 1° C.-30° C.; and a headband configured to secure the conformable body to the subject's forehead.

Also described herein are methods of treating a subject using any of these apparatuses. A method of treating a subject (e.g., to improve or enhance sleep, to improve mood, etc.) may include: applying, by a controller, a control scheme to a plurality of thermoelectric temperature regulators in communication with a skin-facing surface of a conformable body of an apparatus worn on a subject's forehead, wherein the control scheme repeatedly: applies power to the thermoelectric temperature regulators to cool or heat the thermally conductive skin-facing surface in thermal communication with the thermoelectric temperature regulator to a set temperature of between 1-30° C., maintains the set temperature for a dwell time of between 1 and 15 seconds, and reduces or stops power to the thermoelectric temperature regulators until a temperature of the thermally conductive skin-facing surface varies from the set temperature by (e.g., a relax temperature of) between 1-5° C.

Any of these methods may include driving one or more cooling fans to cool the thermoelectric temperature regulators while damping vibration of the one or more cooling fans. The method may also include directing airflow from the one or more cooling fans across (e.g., parallel) to the subject's skin and away from the subject's face.

Any of these methods may include damping vibrations by vibrationally isolating the one or more fans using a mechanical damping member that dampens vibrations between 300 and 600 Hz.

In some variations, the methods may include applying a strap of headband over the subject's head to secure the apparatus the apparatus to the subject's forehead.

The method may include operating the controller from a controller housing that is connected to the plurality of thermoelectric temperature regulators by an elongate, flexible cable.

In any of these methods, the power control scheme may stop power to the thermoelectric temperature regulators until the temperature of the thermally conductive skin-facing surface varies from the set temperature by between 1-5° C. Additionally or alternatively, the power control scheme may dynamically change the dwell time and/or the relax temperature. In some variations, the power control scheme increases the dwell time as the apparatus is continuously operated.

The method may include detecting one or more of the subject's autonomic state and the subject's sleep/wake state, further wherein the controller is configured to adjust power to the thermoelectric temperature regulators based on input from the sensor. For example, detecting one or more of the subject's autonomic state and the subject's sleep/wake state and adjusting power to the thermoelectric temperature regulators based on the subject's autonomic state or the subject's sleep/wake state. In some variations the method may include switching the control scheme to a standby mode based on one or both of the subject's autonomic state and the subject's sleep/wake state.

A method of treating may include: applying, by a controller, a control scheme to cool one or more thermoelectric temperature regulators in communication with a skin-facing surface of a conformable body of a an apparatus configured to be worn on a subject's forehead, so that a temperature of a skin-contacting portion of the apparatus is between about 1° C. and 30° C.; and driving a cooling fan to cool one or more thermoelectric temperature regulators while damping vibration of the fan (e.g., between about 25 Hz and about 800 Hz, etc.). Any of these methods may include directing airflow from the one or more thermoelectric temperature regulators parallel to the skin-facing surface.

A method of treating a subject may include: applying, by a controller, a control scheme to a plurality of thermoelectric temperature regulators in communication with a skin-facing surface of a conformable body of an apparatus worn on a subject's forehead, wherein the control scheme cyclically applies power to the thermoelectric temperature regulators to cool or heat the thermally conductive skin-facing surface in thermal communication with the thermoelectric temperature regulator so that a temperature of the thermally conductive skin-facing surface varies by between 1 and 5 degrees about a set temperature of between 1-30° C.; and directing airflow from one or more cooling fans arranged to cool the plurality of thermoelectric temperature regulators so that the airflow is directed away from the subject's face. For example, directing airflow may comprise directing the airflow so that it is parallel to the subject's skin (e.g., across the subject's skin).

As used herein, a treatment may include both therapeutic and non-therapeutic treatments. Treatments are not limited to medical or clinical treatments, but include any treatment. In addition, a subject may be any subject, and is not necessarily an individual with a medical condition. The subject may be human or animal.

Any of these methods may include maintaining a target temperature of the skin-contacting surface for greater than 15 minutes. In addition, any of these methods may include applying the headband over a subject's forehead.

Any of these methods may include applying a control scheme that comprises cooling the skin-facing surface to a set temperature between 1° C.-30° C., wherein the power control scheme varies the power so that the temperature of the one or more thermally conductive skin-facing surfaces deviates from the set temperature by a relax temperature of between 1-5 degrees C., e.g., over a period of between 7 seconds and 5 minutes.

For example, methods of treating a subject with any of these apparatuses may include: applying, by a controller, a control scheme to control the temperature of the skin-contacting region of the apparatus. For example, the control scheme may include (but is not limited to) a sawtooth control scheme. In some variations, the control scheme may deliver a ramp signal to the one or more thermoelectric temperature regulators, thereby reducing the temperature of the skin-facing surface of the apparatus to a target temperature that is between, e.g., 1° C. and 30° C. As mentioned, the methods may include reducing vibration from the one or more thermoelectric temperature regulators using a vibration-damping material (e.g., a foamed vibration-damping material) that is arranged around the one or more thermoelectric temperature regulators; and/or directing airflow from the one or more thermoelectric temperature regulators perpendicular to the skin-facing surface.

The control scheme may be maintained for 15 minutes or more (e.g., 20 min or more, 25 min or more, 30 min or more, 35 min or more, 40 min or more, 45 min or more, 1 hour or more, 1.25 hours or more, 1.5 hours or more, 2 hours or more, etc.).

Any of these methods may include applying the apparatus to the subject. For example, any of these methods may include applying the apparatus to the subject using a headband, e.g., over a subject's forehead. The conformable body of the apparatus may fit into or be otherwise coupled with the headband.

Any of these methods may include controlling the apparatus using a hand-held housing with one or more controls, as described above. For example, any of these methods may include operating the apparatus from a control unit comprising a housing enclosing the controller, wherein the control unit is connected to the headband by an elongate, flexible cord. In some variations, operating comprises manually adjusting a control on the control unit. Alternatively, the control unit may be self-contained, and may not require adjustment or may be automatically adjusted.

Applying the control scheme may comprise reducing the temperature by a variable or predetermined amount (e.g., by between 1° C. and 30° C.). In some variations, applying the control scheme comprises ramping up or down the power applied to the one or more thermoelectric temperature regulators to reduce the temperature on the patient-facing/patient-contacting surface of the apparatus.

For example, described herein are methods of treating a subject to increase relaxation. A method of treating a subject to increase relaxation may include: applying, by a controller, a control scheme to adjust the temperature of the one or more thermoelectric temperature regulators of the apparatus (e.g., of a conformable body of a wearable, e.g., headband, apparatus) thereby reducing the temperature of the skin-facing surface of the apparatus to a target temperature that is between 1° C. and 30° C. Any of these apparatuses may further reduce vibration. For example, the apparatus may dampen vibrations from the fan or fan subassembly by including a vibration-damping material (e.g., a foam such as an open-cell foam) between the fan/fan subassembly and the body of the apparatus that is worn on the subject's head. For example, a foam vibration-damping material may be arranged at least partially around the one or more fans or fan sub-assemblies. In addition, any of these methods may also include directing airflow from the one or more thermoelectric temperature regulators parallel to the skin-facing surface.

Also described herein are methods of treating a subject to enhance sleep. For example, a method of treating a subject to enhance sleep may generally include: applying, by a controller, a control scheme to adjust the temperature of one or more thermoelectric temperature regulators of an apparatus as described herein. In some variations, the apparatus may include a body having a skin-facing surface that is worn against the subject's head (either directly or through a comfort layer or cover) thereby reducing the temperature of the skin to a target temperature that is between 1° C. and 30° C. As mentioned, any of these methods may include reducing vibration from the one or more thermoelectric temperature regulators using a vibration-damping material (e.g., foam) arranged at least partially around the one or more fans cooling the thermoelectric temperature regulators. The method may also include adjusting the airflow from the one or more thermoelectric temperature regulators parallel to the skin-facing surface.

Also described herein are methods of treating a subject to improve mood. A method of treating a subject to improve mood may generally include: applying, by a controller, a control scheme to control the energy applied to one or more thermoelectric temperature regulators arranged on the apparatus, thereby reducing the temperature of the skin-facing surface of the apparatus to a target temperature that is between, e.g., 1° C. and 30° C. Any of these methods may also include reducing vibration from the one or more thermoelectric temperature regulators using a vibration-damping material. This material may be arranged at least partially around the one or more thermoelectric temperature regulators. Any of these methods may also include directing airflow from the one or more thermoelectric temperature regulators parallel to the skin-facing surface.

In some variations, described herein are methods of treating a subject to reduce or prevent headaches (including, but not limited to, migraines). For example, a method of treating and/or preventing a subject to treat headaches may generally include: applying, by a controller, a control scheme to adjust the temperature of one or more thermoelectric temperature regulators, which may be coupled to the body of the apparatus and in thermal contact with one or more skin-facing surfaces of a conformable body of the apparatus, thereby reducing the temperature of the skin-facing surface of the apparatus to a target temperature that is between, e.g., 1° C. and 30° C. Alternatively or additionally, any of these methods may include reducing or eliminating vibration from one or more fans configured to cool the thermoelectric temperature regulators. The vibration may be dampened using a foamed vibration-damping material that may be arranged at least partially around the one or more thermoelectric temperature regulators. Any of these methods may include directing airflow from the one or more thermoelectric temperature regulators parallel to the skin-facing surface.

As mentioned above, any of these apparatuses may include one or more sensors. The sensor(s) may be configured to collect data from the subject and transit this data (wired or wirelessly) to the controller/processor in the apparatus, or a remote processor that is in communication with the controller in the wearable apparatus. Sensors may include sensors to monitor the operation of the device and/or the effect(s) or status of the subject wearing the apparatus. For example, sensor data may be used by the controller/processor to determine the sleep state of the subject wearing the device (e.g., awake, NREM (stage 1, 2 or 3), REM sleep, etc.), determine if the subject is wearing the apparatus and/or determine the parasympathetic status of the subject. Modulation of the parasympathetic nervous system may be used to reduce sleep onset and maintain sleep. One manner in which the autonomic nervous system can be modulated is through the primitive autonomic nervous system reflex known as the diving reflex. The diving reflex is triggered by immersion of the body in cold water, and is characterized by a reduction in heart rate (HR) due to an increase in cardiac vagal activity, a primary efferent of the parasympathetic nervous system; this is often associated with vasoconstriction of selected vascular beds, due to increased sympathetic output to the periphery. Thus, any of the apparatuses and methods described herein may be configured to induce a diving reflex response in a subject wearing the apparatus by providing cooling to the forehead of the subject (including local, spot cooling). Sensors that may detect the parasympathetic state (e.g., heart rate, heart rate variability, etc.) may therefore also be used to modulate the applied cooling therapy and toggle between cooling and standby temperatures.

Thus, any of these apparatuses and method of using them as described herein may include a sensor configured to detect one or more of the subject's autonomic state and the subject is sleep/wake state. The sensor may be an accelerometer integrated into the conformable body (e.g., detecting body position and/or motion, which may also be used to derive sleep state or simply sleep/awake status). The sensor may be configured to detect one or more of: body movement, respiratory rate, heart rate, electrocardiogram (ECG) signals, and electroencephalogram (EEG) signals.

In general, the controller may be configured to apply the power to cool the thermoelectric temperature regulator to between, e.g., 1° C.-30° C. In some variations, the controller may control the applied temperature during an active cooling phase for a predetermined time period (e.g., 10 minutes to 60 minutes, 10 minute to 45 minutes, etc. or any time between 10 minutes and 3 hours, which may be user selected or automatically determined), or until the apparatus detects that the user is asleep. The apparatus may then toggle into a standby state and may regulate the temperature at a standby temperature (e.g., between 26° C. and 38° C., between 28° C. and 38° C., between 30° C. and 38° C., etc. or within a few degree, e.g., +/−2° C., of body surface/skin temperature). In some variations, the apparatus may apply the cooling in a ramp with decreasing temperature (e.g., decreasing from 30° C. or 25° C.) until the subject experiences a diving reflex, at which point the temperature may be sustained for a predetermined first treatment time period (e.g., 10 minutes to 60 minutes, 10 minute to 45 minutes, etc. or any time between 10 minutes and 3 hours). For example, a controller may be further configured to apply power to cool the thermoelectric temperature regulator to between 1° C.-30° C. when the subject is awake.

In any of these examples, the controller may be configured to apply power to the thermoelectric temperature regulators to maintain a standby temperature, e.g., of between about 18° C.-38° C. (e.g., between about 20-30° C., between about 18-30° C., between about 22-30° C., between about 23-30° C., between about 24-30° C., etc.) during a standby period. The standby period may be user-defined, including selecting a time interval or time of day, or may be detected, e.g., by detecting when the subject is asleep and/or active (e.g., based on an accelerometer sensor data). The standby period may be set to a fixed duration (e.g., x min, where x is 2 min, 5 min, 8 min, 10 min, 12 min, 15 min, 20 min, 30 min, etc.) Thus, the controller may be configured to detect a standby condition, based one or more sensors (e.g., detecting a triggering event, such as movement above or below a trigger level, heart rate, etc.), and/or based on time of day or time the apparatus was actively cooling.

Any of the apparatuses described herein may be battery powered (and/or rechargeable). For example, the apparatus may further comprise a battery. Any of these apparatuses may include a charging circuit and/or a charging antenna configured to recharge the battery from power inductively received by the charging antenna. The charging antenna may be configured to power the controller and the thermoelectric temperature regulators when power is received by the charging antenna.

Any of these apparatuses may include a wireless communications circuit configured to wirelessly transmit and receive to and from the controller.

In general, the controller may be configured to apply energy in a pulsatile manner so that the thermoelectric temperature regulators apply variably cooling to the forehead (e.g., varying between the cooling temperature and a temperature that is slightly higher than the cooling temperature). For example, the energy may be applied in pulses having a pulse duration of less than 360 seconds. As descried above, in particular, these devices may be configured to apply energy to the thermoelectric temperature regulator elements using a control scheme.

Any of these apparatus may be configured for use with a comfort layer (e.g., cover) which may be removably placed over the skin-facing thermal transfer surface. As mentioned, the comfort layer may be removable or non-removable, and may have a thermally transmissive surface configured to be positioned over the skin-facing surface. For example, the cover may be a single-use cover to prevent dirtying of the device when worn and/or to help secure the device to the forehead.

Also described herein are methods of using these apparatuses for applying thermal energy to a subject's forehead to do one or more of: increase relaxation, improve mood and/or enhance sleep. In some variations these methods may switch between an active mode and a standby mode. For example, a method of applying thermal energy to enhance sleep may include: wearing a conformable body on the subject's forehead having a skin-facing surface; applying cooling from a plurality of thermoelectric temperature regulators arranged across the skin-facing surface; sensing one or more subject parameters (e.g., movement, heart rate, blood pressure, etc.) with a sensor on or in communication with the apparatus; determining if a triggering parameter has occurred, and switching between applying power to cool the thermoelectric temperature regulators to a first temperature of between 1° C.-30° C. and a second, warmer, standby temperature of between 20-30 degrees C. when the triggering parameter has occurred.

Any of the methods described herein may include using the thermally-regulated applicator attached to a subject's forehead and applying power, using a controller in the thermally-regulated applicator, to control a plurality of thermoelectric temperature regulators on the thermally-regulated applicator to cool a thermal transfer surface on the thermally-regulated applicator to a first temperature of between, e.g., 1° C.-25° C. for a treatment period and decreasing the power applied to the thermoelectric temperature regulators after the first duration to maintain the thermally-regulated applicator at a standby temperature, e.g., of between about 20° C.-30° C. for a standby period. The method may include cycling between the therapy period and the standby period. The therapy period may have a duration of greater than 10 minutes (e.g., greater than 15 minutes, greater than 20 minutes, greater than 30 minutes, greater than 40 minutes, greater than 45 minutes, greater than 1 hour, etc., including any value between 10 and 120 minutes).

The therapy period may end after a predetermined time period or after a triggering event. For example, the controller may monitoring the subject, but if a trigger event (e.g., based on subject movement) has occurred and the controller may switch to the standby period. The predetermined time period may be greater than 10 minutes (e.g., greater than 15 minutes, greater than 20 minutes, greater than 30 minutes, greater than 40 minutes, greater than 45 minutes, greater than 1 hour, etc., including any value between 10 and 120 minutes).

The standby period may have a duration of greater than 10 minutes (e.g., greater than 15 minutes, greater than 20 minutes, greater than 30 minutes, greater than 40 minutes, greater than 45 minutes, greater than 1 hour, greater than 2 hours, greater than 3 hours, etc., including any value between 10 and 120 minutes).

### BRIEF DESCRIPTION OF THE DRAWINGS

FIG. 1A is a schematic illustration of the skin-contacting surface of one variation of an apparatus for applying thermal energy to a subject's forehead using a thermoelectric material as described herein.

FIG. 1B is a schematic illustration of the opposite side of the apparatus of claim 1A.

FIGS. 2A-2C illustrate schematic partially transparent views through variations of apparatuses for applying thermal energy to a subject's forehead using a thermoelectric material.

FIG. 3A is an exemplary block diagram illustrating an apparatus for applying thermal energy to a subject's forehead.

FIG. 3B is another exemplary block diagram illustrating an apparatus for applying thermal energy to a subject's forehead using a thermoelectric material.

FIG. 4 is an example of an apparatus for applying thermal energy to a subject's forehead.

FIG. 5 illustrates a subject wearing an apparatus for applying thermal energy to a subject's forehead using a thermoelectric material.

FIG. 6 is an example of an apparatus for applying thermal energy to a subject's forehead using a thermoelectric material that may be batteryless and/or may be charged while sleeping using an inductive charger positioned beneath or near the sleeping subject.

FIG. 7 illustrates wireless communication between an apparatus for applying thermal energy to a subject's forehead using a thermoelectric material, a remote processor (e.g., smartphone), and/or one or more sensors for monitoring subject parameters (including wearable sensors and/or bedding sensors).

FIG. 8A shows the inner (skin-facing) surface of an exemplary prototype of an apparatus for applying thermal energy to a subject's forehead using a thermoelectric temperature regulator (TTR), including eight TTR cooling surfaces arranged across the skin-facing side of the device, for contacting the skin (although a thermal spreader may be used to distribute the applied cooling/warming).

FIG. 8B shows the outer surface of the apparatus of FIG. 8A, showing the opposite side of the thermoelectric temperature regulators, including heat sinks.

FIG. 9 illustrates one example of an apparatus as described herein applied to a subject's forehead.

FIG. 10 is a front perspective view of a prototype apparatus as described herein.

FIG. 11 is side perspective view of the device of FIG. 10.

FIG. 12 is a back view of the device of FIG. 10.

FIG. 13 is a side view of the device of FIG. 10.

FIG. 14 is a top perspective view of a system including a body configured to be worn on the subject's head with one or more thermoelectric temperature regulators, and a hand-held control unit connected by a flexible cable.

FIG. 15A is an example of an apparatus as described herein configured to emit air (e.g., from cooling the thermoelectric temperature regulator(s)) up, towards the crown of the wearer's head, and laterally (toward the wearer's ears).

FIG. 15B is an example of an apparatus as described herein configured to emit air down, e.g., towards the subject's lower face.

FIG. 16A is one example of a schematic of a waveform that may be used as a control scheme for controlling one or more thermoelectric temperature regulators in the apparatus.

FIG. 16B is another example of a schematic of a waveform that may be used as a control scheme for controlling one or more thermoelectric temperature regulators in the apparatus.

FIG. 17 is a partially exploded view of a first prototype of an apparatus for thermally regulating a patient's forehead, that includes a conformable body with multiple thermoelectric temperature regulators, cooling fans and a comfort layer having thermal transfer windows and a removable cover assembly.

FIG. 18 is a side view of the apparatus of FIG. 17 without a comfort layer or removable cover assembly attached.

FIG. 19 illustrates one example of a skin-facing side of a conformable body of an apparatus as described herein, similar to the apparatus shown in FIG. 17.

FIG. 20 shows one example of a thermoelectric regulator assembly as described herein including a TEC and a fan sub-assembly hosed housed within a housing.

FIG. 21 shows a top view of a conformable body of an apparatus as described herein, without a removable cover assembly, showing the outer housing enclosing the fan sub-assembly; the fan is shown floating within the housing with a foam material at least partially surrounding the fan.

FIG. 22 is another top view of the conformable body of the apparatus of FIG. 21 showing the conformable body bending in its long axis.

### DETAILED DESCRIPTION

In general, described herein are apparatuses (e.g., a device and/or a system) for treating a subject. These devices may be used to improve and/or enhance sleep (including, but not limited to treating insomnia, or to generally improve and/or enhance healthy sleep even in non-insomniac subjects), improve or enhance mood, and/or to enhance relaxation. These apparatuses are configured to applying a thermal therapy to the subject's forehead area and may include one, or more preferably a plurality of, e.g., two, three, four, five, six, seven, eight, nine, ten, etc. thermoelectric temperature regulators, a controller applying power to regulate the temperature of the thermoelectric temperature regulators, a body portion, which may be flexible to conform to the subject's head, and a strap to hold the device on the subject's head. The apparatus may also include a power source, such as a battery or capacitive power source, and/or power control circuitry; in some variations the device may be attached to a wall power outlet for operation and/or charging. The apparatus may include one or more sensors, or it may be configured to (e.g., wirelessly) communicate with one or more sensors that may provide information about the operation of the device (e.g., temperature, airflow, movement, etc.), and/or the status of the subject (e.g., subject's sleep state/sleep stage, skin temperature, core temperature, ECG, heart rate, e.g., heart rate variability, body movement, body/head position, body and/or ambient temperature, or the like.

Any of the apparatuses described herein may be specifically adapted for use with a sleeping subject. Thus, these apparatuses may be configured to operate without requiring a subject's input, including providing additional power to the device. As will be described in greater detail below, the controller may be configured to run the thermoelectric temperature regulators in a thermal profile that enhances sleep latency onset (the time to fall asleep) and/or sleep maintenance (sustaining sleep) in a manner that conserves the power.

Any of the apparatuses described herein may apply thermal therapy to the subject. Thermal therapy is defined as the application of a constant or varying temperature between 0° C. and 40° C. (e.g., between a lower temperature of 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C. and an upper temperature of 40° C., 39° C., 38° C., 37° C., 36° C., 35° C., 34° C., 33° C., 32° C., 31° C., 30° C., 29° C., 28° C., 27° C., 26° C., 25° C., 24° C., 23° C., 22° C., 21° C., 20° C., 19° C., 18° C., 17° C., 16° C., 15° C., etc. where the lower range is always less than the upper range), for example between 10° C. and 15° C., between 10° C. and 20° C., etc. to all or part of the forehead area over the frontal cortex.

These apparatuses may be referred to as apparatuses for applying thermal energy to a subject's forehead using a thermoelectric temperature regulator comprising a thermoelectric material. The thermoelectric temperature regulators material may be any appropriate thermoelectric material. Generally, a thermoelectric material may be given its ordinary meaning in the art and refers to materials in which a temperature change is generated at a surface of the material upon application of an electric potential (e.g., voltage and corresponding current), in accordance with the thermoelectric effect, often referred to by other names such as the Peltier, Thomson, and Seebeck effects. While a portion of the description herein describes thermoelectric materials, the present disclosure is not limited to thermoelectric materials, and other thermal adjustment apparatuses may be employed where appropriate. Non-limiting examples of suitable thermoelectric materials may include columns of p-type and n-type doped semiconductor materials, bismuth chalcogenides (e.g., Bi₂Te₃, Bi₂Se₃), lead selenide, Si—Ge alloys, skutterudites (e.g., including the formula LM₄X₁₂, wherein L is a rare earth metal, M is a transition metal, and X is a metalloid), or any other suitable thermoelectric materials.

The dimensions of the thermoelectric temperature regulators described herein may be appropriate so that the apparatus can both generate the appropriate temperature profile, may cover a sufficiently large region of the subject's forehead, and may remain flexible enough so that the apparatus, typically including an array of thermoelectric temperature regulators (also referred to herein as thermoelectric modules, thermoelectric elements or thermoelectric cooler/heaters), may conform comfortably to the subject's forehead. A thermoelectric temperature regulator may have any suitable thickness. For example, in some embodiments, the thickness may be selected such that the thermoelectric temperature regulator(s) may be comfortably held against the forehead. In some embodiments, the thickness of each of the thermoelectric materials may be between about 1 millimeter and about 5 millimeters (e.g., between about 1 millimeter and about 3 millimeters). Other thicknesses are also possible. Each of the thermoelectric materials, or modules that include the thermoelectric temperature regulator(s), may have a largest average cross-sectional dimension of between about 10 mm and about 4 cm (e.g., between about 30 mm and about 500 mm). Other average cross-sectional dimensions are also possible. Those skilled in the art would be capable of selecting an appropriate size for the thermoelectric material based upon the configuration of the device.

A thermoelectric temperature regulator may be provided in any suitable configuration. For example, a module may include thermoelectric materials sandwiched between ceramic plates, in some cases, for protection and support, as well as to provide thermal conductivity to the surface of the skin. The thermoelectric temperature regulator may be configured to be directly applied to the skin, or a thermally conductive intermediary material (e.g., a reusable or disposable cover, layer, etc.) may be interposed between the subject's skin and the surface of the thermoelectric temperature regulator. The material forming the outer skin-contacting surface of the apparatus may be an outer surface of the thermoelectric temperature regulator and may be washable. The outer surface may be a metallic (e.g., aluminum, stainless steel, etc.). The back (outward-facing) side of the apparatus in some variations may include one or more structures to help dissipate heat, including heat sinks, fans, or the like. Further, in some variations, the apparatus may be configured to include a soft material (e.g., a filling, such as a foam and/or cushioning material), which may also help distribute the waste heat, and may make the apparatus more comfortable for use while sleeping.

In general, the apparatuses described herein may not cover the eyes of the subject wearing them. Alternatively in some variations, an eye-covering portion may also be included.

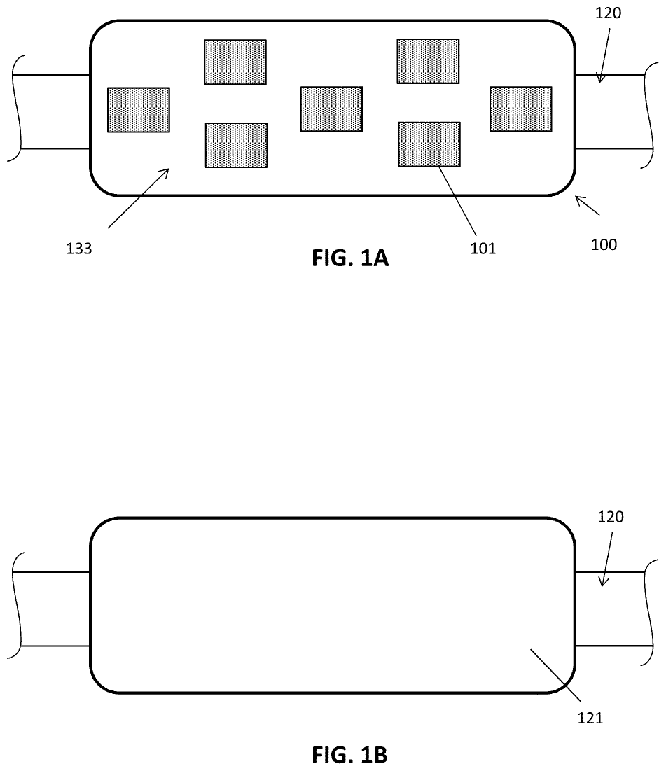

Any of these apparatuses may include a thermally insulative material positioned so as to surround and/or insulate the cooling portion of the thermoelectric temperature regulator (e.g., the skin-facing side, worn against the subject's skin) from the warming side of the thermoelectric temperature regulator. For example, an outward-facing surface of the apparatus maybe partially covered by an insulative material; regions coupled to the thermoelectric temperature regulator heating side may be coupled to a heat sink material to dissipate waste heat. Alternatively, all or a portion of the outward-facing surface of the device (see, e.g., FIG. 1B) may be exposed and/or may include a heat sink and/or fan.

In general, the apparatuses described herein may be thin, lightweight wearable devices adapted to be worn on a subject's head while awake or while sleeping to provide cooling and/or heating to the specific region of the wearer's forehead. These apparatus may be flexible, and do not require a circulating cooling fluid.

As mentioned the devices may use one or more thermoelectric temperature regulators, which may be configured as thermoelectric coolers (e.g., TECs) that may be placed directly on the skin covering all or part of the forehead area. The use of a thermal spreader material can be used to help distribute the TEC temperature over a larger area than covered by the TECs. Thermal spreader materials are widely known in thermal management.

A conformable material may be used as an interface layer between the thermoelectric temperature regulators and the skin to improve comfort from the rigid thermoelectric temperature regulators surface. This conformable material may or may not act as a thermal spreader.

In some variations, the thermoelectric temperature regulators can be used in conjunction with known phase change materials or evaporative cooling to provide the desired temperature profiles.

Apparatuses and methods may be included to control the waste heat generated by the thermoelectric temperature regulators. As mentioned, one or more heat sinks and/or fans may be used. In addition, the apparatus may be adapted to provide evaporative cooling. For example, the apparatus may include a reservoir of an evaporative cooling material (e.g., water, alcohol, etc.) that may be released during use small amounts onto an evaporative cooling surface to increase heat transfer. This release may be temperature dependent. For example, the apparatus may include a temperature sensor to determine the temperature of the non-skin contacting side and may provide evaporative cooling by release of metered amounts of fluid material to allow evaporative cooling when the temperature is above a threshold temperature (e.g., greater than 40° C., greater than 45° C., greater than 50° C., etc.).

Any of the forehead-cooling apparatuses and methods described herein may include an evaporative heat sink for removing waste heat from the apparatus. For example, these apparatuses may include a heat sink and a wetting material disposed on the heat sink that is configured to hold a desired amount of cooling liquid (e.g., water), without adding an excessive heat transfer resistance or heat capacity. Alternatively or additionally, the apparatus may cycle the electrical power supplied to the thermoelectric temperature regulators. Thus, rather than hold the thermoelectric temperature regulators at a constant temperature, the apparatus may take advantage of the fact that the temperature receptors on the surface of the forehead, and particularly the cooling sensors, may respond to the change in the temperature. Thus, these apparatuses may provide efficient cooling (including, but not limited to inducing sleep onset and maintenance) by cycling to a temperature (e.g., a low temperature of between 0° C. and 25° C., e.g., between 10° C. and 20° C., between 10° C. and 18° C., between 10° C. and 17° C., etc.) from a temperature that is slightly warmer (e.g., greater than 5° C. warmer, greater than 6° C. warmer, greater than 7° C. warmer, greater than 8° C. warmer, greater than 9° C. warmer, greater than 10° C. warmer, between 2° C. and 15° C. warmer, between 3° C. warmer and 14° C. warmer, between 4° C. warmer and 13° C. warmer, etc., including fractional degrees C.).

Thus, any of these apparatuses may include a body having a skin-contacting surface configured to be worn flush against the surface of a subject's forehead that includes a plurality of thermoelectric temperature regulators (TEC modules), one or more heat sink thermally coupled to each of the thermoelectric temperature regulators, typically on the side opposite from the skin-facing side, to dissipate waste heat. In some variations the apparatus may also include a fan and/or a wetting material in thermal communication with the heat sink(s). Each of these apparatus may also include or be configured to communicate with a controller for controlling the temperature of the thermoelectric temperature regulators. The controller may also receive input from one or more sensor(s), including sensors for detecting the sleep state of the user, or for detecting if the user is experiencing a diving reflex, which may be associated with enhancing sleep by reducing sleep onset or sustaining sleep. The controller may also coordinate the cooling of the forehead using the apparatus by cycling the thermoelectric temperature regulators in accordance with a duty cycle, for example, a duty cycle greater than about 10% during an active cooling period. The controller may also switch from an active cooling period in which the forehead is cooled to the first target temperature of, e.g., between 10° C. and 25° C., or any other appropriate cooling range) and a standby temperature of between 26° C.-38° C., and/or a temperature close to the subject's normal skin temperature (which may be empirically determined or determined by sensing from the forehead or other regions). Switching between an active cooling temperature and a standby temperature may maintain the battery life while preventing sleep disruption in variations having a battery.

The apparatuses described herein may be configured to operate (and/or may incorporate any of the features) as described in: U.S. patent application Ser. No. 15/597,078, titled “WEARABLE THERMAL DEVICES AND METHODS OF USING THEM FOR TREATMENT OF SLEEPING AND NEUROLOGICAL DISORDERS” and filed on May 16, 2017, which claims priority to U.S. provisional patent application No. 62/337,721, filed on May 17, 2016, and titled “TETHERLESS WEARABLE THERMAL DEVICES AND METHODS OF USING THEM FOR TREATMENT OF SLEEPING DISORDERS.” These apparatuses and methods may also be configured to operate (and/or incorporate any of the features) as described in one or more of: U.S. patent application Ser. No. 13/019,477, filed on Feb. 2, 2011 (now U.S. Pat. No. 8,425,583), titled “METHODS, DEVICES AND SYSTEMS FOR TREATING INSOMNIA BY INDUCING FRONTAL CEREBRAL HYPOTHERMIA;” U.S. patent application Ser. No. 13/868,015, filed on Apr. 22, 2013 (now U.S. Pat. No. 9,089,400), titled “METHODS, DEVICES AND SYSTEMS FOR TREATING INSOMNIA BY INDUCING FRONTAL CEREBRAL HYPOTHERMIA;” U.S. patent application Ser. No. 14/749,590, filed on Jun. 24, 2015, titled “METHODS, DEVICES AND SYSTEMS FOR TREATING INSOMNIA BY INDUCING FRONTAL CEREBRAL HYPOTHERMIA;” U.S. patent application Ser. No. 11/788,694, filed on Apr. 20, 2007 (now U.S. Pat. No. 8,236,038), titled “METHOD AND APPARATUS OF NONINVASIVE, REGIONAL BRAIN THERMAL STIMULI FOR THE TREATMENT OF NEUROLOGICAL DISORDERS,” U.S. patent application Ser. No. 14/938,705, filed on Nov. 11, 2015, titled “APPARATUS AND METHOD FOR MODULATING SLEEP;” U.S. patent application Ser. No. 12/288,417, filed on Oct. 20, 2008 (now U.S. Pat. No. 9,492,313), titled “METHOD AND APPARATUS OF NONINVASIVE, REGIONAL BRAIN THERMAL STIMULI FOR THE TREATMENT OF NERUOLOGICAL DISORDERS;” and U.S. patent application Ser. No. 14/341,642, filed on Jul. 25, 2014 (now U.S. Pat. No. 9,211,212), titled “APPARATUS AND METHOD FOR MODULATING SLEEP.”

In variations in which a wetting material is included to help remove (by evaporation) the excess/waste heat, the apparatus may include a refillable reservoir of fluid and/or a supply line connecting the wetting material and the reservoir. The reservoir may include a sponge or other porous material. The supply line may include a wicking material to convey a liquid from the reservoir to the wetting material; alternatively the reservoir (e.g., sponge) may be connected directly to the wetting material. The heat sink may be any appropriate thickness (e.g., from about 1 mm to about 15 mm). A binding layer may secure the wetting material to the heat sink (e.g., the wetting material may be disposed between the heat sink and the binding layer). The binding layer may be wrapped around an outer edge of the heat sink. Alternatively or additionally, the wetting material may be a sponge material that holds the fluid therein. The heat sink may include an etched surface and/or a contoured surface. The contoured surface may form a bend having a bend angle in a range from about 0 degrees to about 90 degrees. In one embodiment, the wetting material includes an antimicrobial agent. In another embodiment, the wetting material includes a hydrophilic material. A thickness of the wetting material may be from about 1 mm to about 3 mm. The wetting material may include tissue and/or cotton.

As will be described in greater detail herein, the apparatus may include a plurality of thermoelectric temperature regulators distributed along the inner surface of the apparatus. Although a thermally conductive ‘spreading’ material may be used to distribute the cooling from the thermoelectric temperature regulators along the skin-contracting side of the apparatus, in some variations the apparatus does not include an additional spreader, but instead applies spots of cooling at the desired locations. These spots may be discrete or diffuse.

In general, the controller for any of the apparatuses described herein may include control logic (hardware, firmware, and/or software) for controlling the application of energy to the thermoelectric temperature regulators (including feedback) to regulate the temperature of the apparatus. For example, control logic may be employed to power the TECs to reduce waste heat or reduce power consumption. The duty cycle of power applied to the TECs can be varied in various ways to reduce waste heat and reduce power consumption. In particular, any of the apparatuses described herein may be configured to apply pulsed cooling/heating, as will be described in greater detail below. Alternatively, stead-state heating and/or cooling may be applied.

The thermoelectric temperature regulators may be powered in any appropriate manner. The apparatus, including the thermoelectric temperature regulators, can be powered by a control unit placed on a table near the subject and power can be supplied by an electrical wire from the control unit to the TECs on the subject (e.g., via a wall power source or line). The apparatus, including the thermoelectric temperature regulators, could be powered by control unit that includes a battery located on the subject's body and wired to the TECs. The battery could be removed from the subject worn control unit and recharged in a remote charging station. Multiple batteries could be used whereby batteries could be charging while others are used to power the TECs and exchanged when needed.

In some variations, the apparatus may be batteryless or may include batteries that are charged during use. For example, the device may be powered and/or the batteries could be charged while in use by induction charging. For example a coil may be located on the subject and wired to the battery charging circuit and/or a charging coil may be included as part of the apparatus. A second induction charging coil for applying the power may be located in proximity to the first coil when the apparatus is in use and/or charging, to induce induction charging of the system. As mentioned, the induction charging could provide all of the power to the TECs without the need for batteries. For example, an induction coil may be included in the bedding (e.g., mattress, pillow, etc.), headboard, bedframe, nightstand, wall-mount, etc. and positioned so that the apparatus may receive power when the subject is wearing the apparatus and lying in the bed.

The control unit may control power to the thermoelectric temperature regulators by any number of commonly known means such as pulse width modulation or simple analog circuits. The temperature of the thermoelectric temperature regulators and or the skin temperature and or the interface material can be monitored by one or more sensors and controlled by a microprocessor and software or it could be controlled by analog circuits. In one variation the power supplied to the thermoelectric temperature regulators is controlled by a time dependent algorithm. In one variation the power supplied to the thermoelectric temperature regulators is controlled by a bio feedback algorithm such as sleep staging information gathered by various sensors/software commonly used to determine sleep staging. In one variation, the power could be conserved by reducing the power to the thermoelectric temperature regulators when sensors indicate the subject is in stage 1 or stage 2 sleep and could be increased when it is detected the subject is no longer in the desired sleep stage. Other measurements, such as body movement or EEG signals may be used for TEC control.

FIG. 1 is a schematic illustration of one example of an apparatus 100 as described herein configured as a forehead cooling and/or heating. The apparatus 100 including a plurality of thermoelectric temperature regulators 101 thereon. In some variations fewer (e.g., just 1, just 2, etc.) or more (e.g., 8, 9, 10, etc.) may be used and may be positioned thereon in any arrangement, including arrangements specifically configured to preferentially modulate a parasympathetic and/or sympathetic response. The thermoelectric temperature regulators are shown as rectangular, however, they may be any shape, including round, oval, square, interlocking, etc. In FIG. 1, the body of the apparatus is shown as rectangular, however, it may be any shape, and in particular, may be a shape that is configured specifically for placement on the forehead. The body may be a conformable body 133 configured to flex, bend, and/or otherwise conform to the curvature of the subject's forehead. The thermoelectric temperature regulators may be arranged along the body so that they contact the non-hairy skin of the forehead on most (e.g., average) subjects. The body of the device may be shaped as a curved or curveable shape to match the curvature of the forehead. The apparatus may include a strap 120 or other securement to hold the device over the subject's forehead.

In FIG. 1A the apparatus has 7 thermoelectric temperature regulators arranged across the surface. The thermoelectric temperature regulators are separated by space, and the thermoelectric temperature regulators may be positioned on a substrate so that the body can be flexibly positioned over the forehead.

As mentioned above, the thermoelectric temperature regulators, during use, may be configured to be positioned directly adjacent to the surface of the user's skin. The apparatus may include multiple thermoelectric temperature regulators positioned at the surface of the skin, and may optionally include a thermally conductive material (e.g., heat sink) 121 located on the opposite side of the apparatus (e.g., the outward-facing side when worn), as shown in FIG. 1B, in a manner that covers the thermoelectric temperature regulators. A thermally conductive material may dissipate heat to and/or from the thermoelectric temperature regulator(s), as desired. The thermally conductive material may include any suitable material, such as metal (e.g., aluminum, copper, stainless steel, etc.), thermally conductive polymer, porous ceramic, or another appropriate material. In some variations, rather than a thermally conductive material, a thermally insulative material may be located on the side of the thermoelectric temperature regulator opposite the skin, covering the thermoelectric temperature regulator(s). Covering the thermoelectric temperature regulator(s) with a thermally insulative material may enhance the effects of thermal pulsing at the surface of the skin, when pulsing is used to apply cooling or heat. It is not required for the thermoelectric temperature regulators to be covered by a thermally conductive or insulative material. For example, a thermal dissipation apparatus may be spaced from or located adjacent to the thermoelectric temperature regulators, without covering the thermoelectric temperature regulators. Alternatively or additionally, the thermally conductive or insulative material may be arranged so as to cover a portion of the thermoelectric temperature regulators.

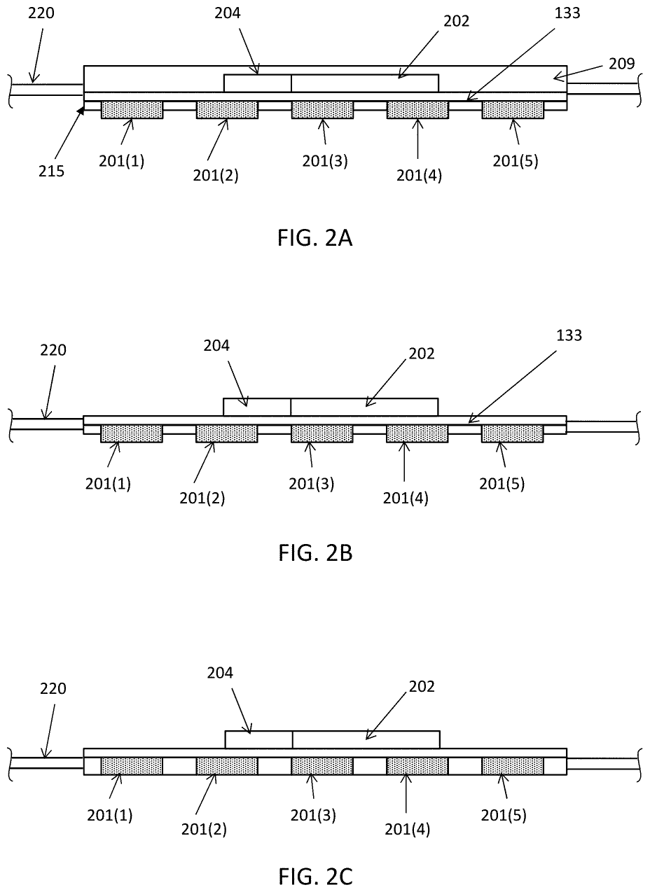

The apparatus may be connected to a power source 204 (e.g., battery, plug-in outlet, etc.) and a controller 202, for applying appropriate signals to the thermoelectric, for manipulating the temperature at the surface of the skin. For example, FIGS. 2A-2C illustrate variations of apparatuses shown in side, partially transparent views, so that arrangement of parts is visible.

In FIG. 2A, the apparatus includes a plurality of thermoelectric temperature regulators 201(1), 201(2), 201(3), 201(4), 201(5), that are arranged along the bottom, skin-facing side of the apparatus. In this example the bottom of the thermoelectric temperature regulators may be configured to contact the skin of the forehead; alternatively a cover that is thermally transmissive or that includes a thermally transmissive window, may be worn over the apparatus so that the thermoelectric temperature regulators can contact the skin. As shown in FIG. 2A, the thermoelectric temperature regulators project slightly from the bottom of the apparatus, but are arranged in a substrate 215, 133. The substrate may be flexible and may include electrical connections between the thermoelectric temperature regulators, the controller 202 and the power supply 204. The connectors may be wires or traces, and they may be formed on or in the flexible substrate(s). In FIG. 2A, the back side of the thermoelectric temperature regulators, facing away from the subject when worn, are shown covered in a material, such as a thermally conductive material as discussed above. This may protect the apparatus while allowing rejection of unwanted heat, e.g., via a heat sink.

The controller may have one or more inputs and/or outputs to accommodate user control of the device in a suitable and convenient manner. For example, the controller may be controlled by an ‘on’ button, an ‘off’ button, a timer, a mode set, etc.

FIG. 2B shows an example of an apparatus similar to that shown in FIG. 2A. In this example, the apparatus is nearly the same as shown in FIG. 2A, but does not include the (optional) covering 209 shown in FIG. 2A. The conformable body 133 of the device may be pre-shaped to fit a generic forehead (e.g., having a concave shape) and/or may be flat and configured to bend and conform. Any of these apparatuses may include cuts, slits, hinged regions or other structures to enhance bending or conforming to the head (forehead) shape. In any of these apparatuses the body may be formed at least in part of a flexible and/or stretchable fabric or other material. Similarly, FIG. 2C is another example in which the apparatus does not include a cover as shown in FIG. 2A. In this example, the thermoelectric temperature regulators are embedded and flush with the substrate. This may allow better surface contact between the target, e.g., skin, and the thermal transfer surface connected to the TEC. Additional elements may also be included, such as one or more sensors (e.g., temperature sensors, accelerometers, etc.), an inductive charging antenna (e.g., coil), charging circuitry, a clock, a display and/or indicator lights, etc.

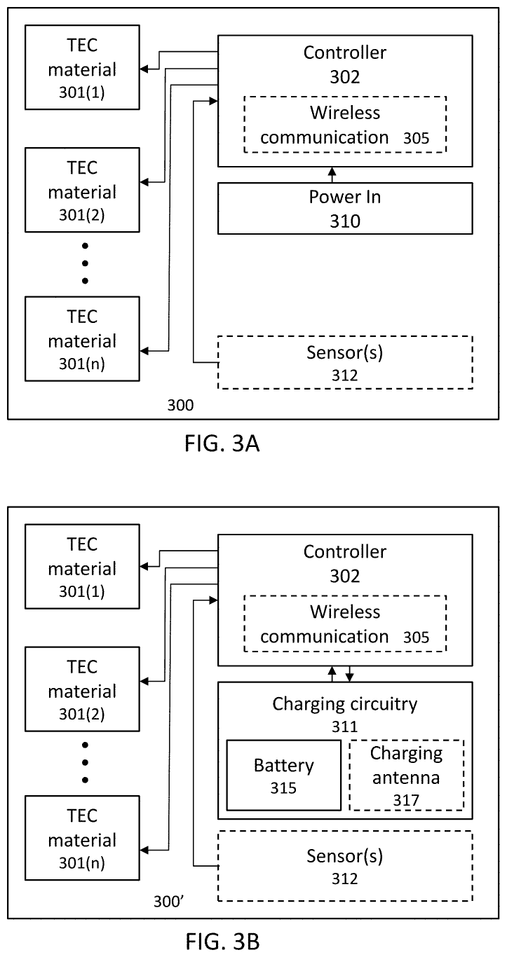

For example, FIG. 3A-3B schematically illustrate examples of apparatuses 300. In FIG. 3A, the apparatus includes a plurality of n thermoelectric temperature regulators 301(1), 301(2), . . . 301(n), each of which are controlled by a controller 302. The controller may communicate with (e.g., transmit to, receive from) a remote processor, such as a smartphone, laptop, computer, or dedicated control device. In FIG. 3A an optional wireless communication sub-system 305 may be included. This sub-system (which may be integrated with and/or part of the controller 302) may include a communications antenna, e.g., for RF, near field, Zigbee, Bluetooth (near field Bluetooth), etc. In some variations the apparatus may also include one or more sensors 312. The sensors may be biological/physiological sensors , such as temperature sensors, oxygenation sensors, ECG sensors, galvanic skin response sensors, etc. The sensors may be integrated into the device, or they may be separately coupled to the user (e.g., worn as part of a bandage, etc.).

The apparatus (system) of FIG. 3B is similar to what is shown in FIG. 3A, but also includes charging circuitry 311. As mentioned, the applicator may be charged inductively either before, during or after use. As mentioned, in some variations the charging circuitry may also be used for communications, e.g., for telemetry, and/or may be integrated into the controller circuitry 302.

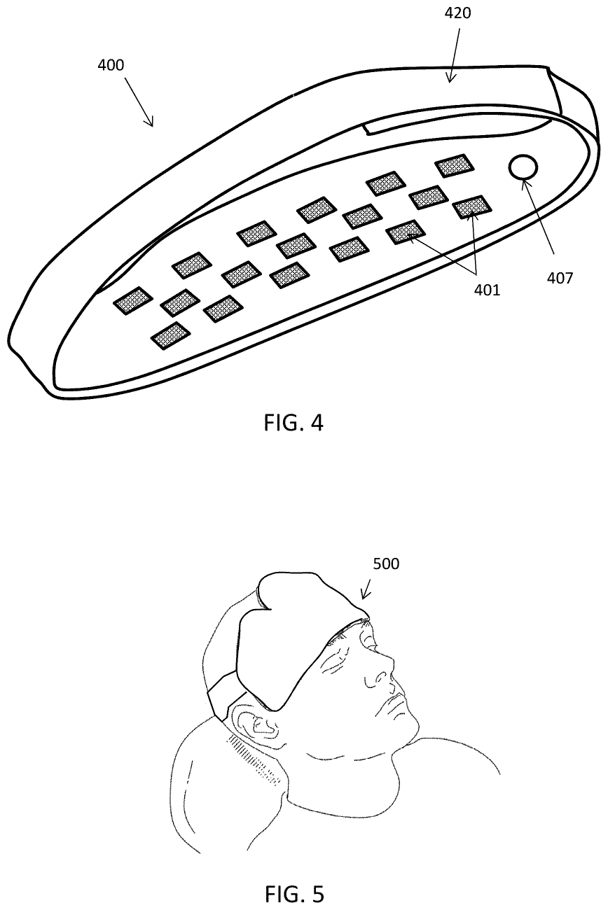

FIG. 4 is an example of an apparatus 400 shown as integrated with a holdfast 420, shown in FIG. 4 as a strap, for securing the device to the head and over the forehead, as shown in FIG. 5. The holdfast may also or alternatively be an adhesive (e.g., releasable skin adhesive), cap, headband, or the like. Returning to FIG. 4, the apparatus includes a plurality of small and thin thermoelectric temperature regulators 401 arranged along the internal skin-contacting surface. Any appropriate strap may be used, and it may be adjustable to fit different sizes of heads. The strap in FIG. 4 may be secured by Velcro and/or one or more attachments (e.g., snaps, buttons, buckles, etc.) to the head. In FIG. 5, a subject is shown wearing an apparatus 500 and lying down on a bed.

Any of these apparatuses may include one or more sensors 407 which may be integrated into the forehead applicator 400 or separate from it. The apparatus may also include the controller (e.g. one or more processors, not visible in FIGS. 4 and 5) and one or more batteries, including rechargeable batteries, as discussed above.

All of the elements of the apparatuses described herein, i.e., the thermoelectric temperature regulators, thermally conductive material, power source and controller, may be suitably held together by an appropriate band. The band may be flexibly adjustable so as to allow for the thermoelectric temperature regulators to be comfortably and suitably positioned against or otherwise adjacent the surface of the skin such that thermal energy (e.g., thermal pulses) generated by the thermoelectric temperature regulators are effective to provide the subject with a preferred thermal sensation (cooling to between 0° C.-25° C., etc.). For some embodiments, the band may exhibit relatively rigid mechanical behavior, providing support for the overall device. It can be appreciated that the band or strap may have any suitable structure and, in some cases, may have stylistic aspects which may lend the device to be worn over the head and face (e.g., forehead) while sleeping or preparing for sleep, or during waking hours. The strap may include any suitable material, such as, but not limited to, metal, plastic, rubber, leather, synthetic leather, or combinations thereof.

As mentioned, the thermoelectric temperature regulators may be positioned directly adjacent to a surface of the user's skin, in accordance with aspects of the present disclosure, the thermoelectric temperature regulator(s) are not required to be in direct contact with the user's skin; for example, an additional layer (not shown in the figures) may be placed between the thermoelectric temperature regulator(s) and the surface of the skin. For example, a thermally conductive (or selectively/regionally insulative) layer, a protective layer, a support layer (e.g., for added comfort), or another appropriate material.

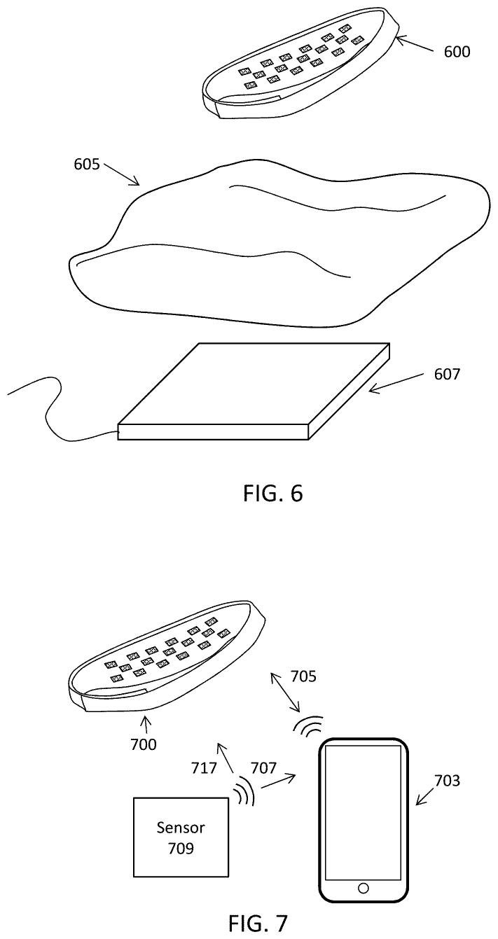

FIG. 6 illustrates an apparatus configured as a system that includes any of the forehead-wearable applicators 600 having a plurality of thermoelectric temperature regulators described above, which includes an antenna for receiving power from a charging pad 607. The charging pad may be integrated into the bedding (e.g., pillow 605, mattress, etc.), bed, headboard, etc. or it may be placed near the subject's head so that the forehead-wearable applicator may charge an apparatus and/or provide power directly so that it may be used without a battery.

FIG. 7 illustrates another example of an apparatus 700 (configured as a system) that also communicates with a separate, external to the apparatus, computing device, such as a smartphone 703 or the like. The smartphone may receive monitoring information and may store it and/or process it. In FIG. 7, the apparatus may also include one or more sensors 709. The sensors may be integrated into the applicator 700, or they may be separate, as shown in FIG. 7. Alternatively or additionally, the sensors may be body-worn sensors or sensors present in the subject's garments, bedding (sheets, mattress, pillow, sleepwear, etc.). Multiple sensors may be used and may provide this information to the controller of the apparatus.

In general, the controller may include a processor that may determine when and how much energy to apply to the thermoelectric temperature regulators to apply cooling or heating to the forehead through the device. In some variations, as shown in FIG. 7, the apparatus may operate in conjunction with a remote processor such as a smartphone 703. Thus, in some variations the processor of the smartphone may be used to determine the timing and/or energy applied to the thermoelectric temperature regulators.

In operation, the apparatus may be configured to apply continuous energy or intermittent energy to the thermoelectric temperature regulators. Alternatively or additionally the apparatus may be configured to generate a series of thermal pulses in succession by applying pulsatile energy to the apparatus. This thermal pulsing may result in an enhanced thermal sensation for a user which may require lower energy than constantly applied power, and yet may activate the thermal neural receptors (e.g., cold receptors) on the forehead comparable to continuously applied temperature.

A thermal pulse may include a transient, reversible temperature change of the thermoelectric temperature regulator, where the temperature changes from an initial temperature to another temperature, quickly followed by a return temperature change back to the initial temperature, or a temperature close to the initial temperature. The time course may be between, for example, 1 second and 360 second (e.g., 1 second and 120 seconds, etc.). A thermal pulse may include a first temperature adjustment at a surface from a first temperature to a second temperature (e.g., at an average rate of 0.1°-10.0° C./sec), and a second temperature adjustment at the surface from the second temperature to a third temperature (e.g., also at an average rate of 0.1°-10.0° C./sec). In such a thermal pulse, the difference in magnitude between the first temperature and the third temperature may be less than 25% of the difference in magnitude between the first temperature and the second temperature. Further, in some cases, the magnitude of the first average rate may be greater than the magnitude of the second average rate.

The pulsatile stimulation described herein may result in a comparable effect compared to steady-state, e.g., constant applied temperature and/or electrical signal modes, so as to maintain long-time scale applications of heating or cooling. For example, thermal pulses may result in continuous thermal stimulation for the human skin. Varying the temperature at the surface of the skin by generating thermal pulses may give rise to a heating or cooling effect that is perceived by the individual that are greater than or equal to those perceived at steady state.

Any of the apparatuses described herein may also be configured to apply energy to maintain the apparatus in a neutral or standby mode in which the apparatus is cooled and/or heated to a neutral temperature (e.g., skin temperature) when not actively cooling/heating, to prevent discomfort when sleeping. For example, the apparatus may be actively used prior to sleeping by maintaining the temperature, e.g., a therapeutic temperature of between 0° C.-25° C. (e.g., between 10° C. and 15° C., etc.). Once the subject wearing the apparatus on their forehead falls asleep, the apparatus may, detecting that the subject is asleep, adjust the temperature to a neutral temperature (e.g., 36° C.-37° C.) while the subject remains asleep. This neutral temperature may keep the device, which may otherwise insulate the head and lead to discomfort, from potentially rousing the subject from sleep. Using a sleep-activated neutral mode as described herein may also help conserve battery power.

### EXAMPLE

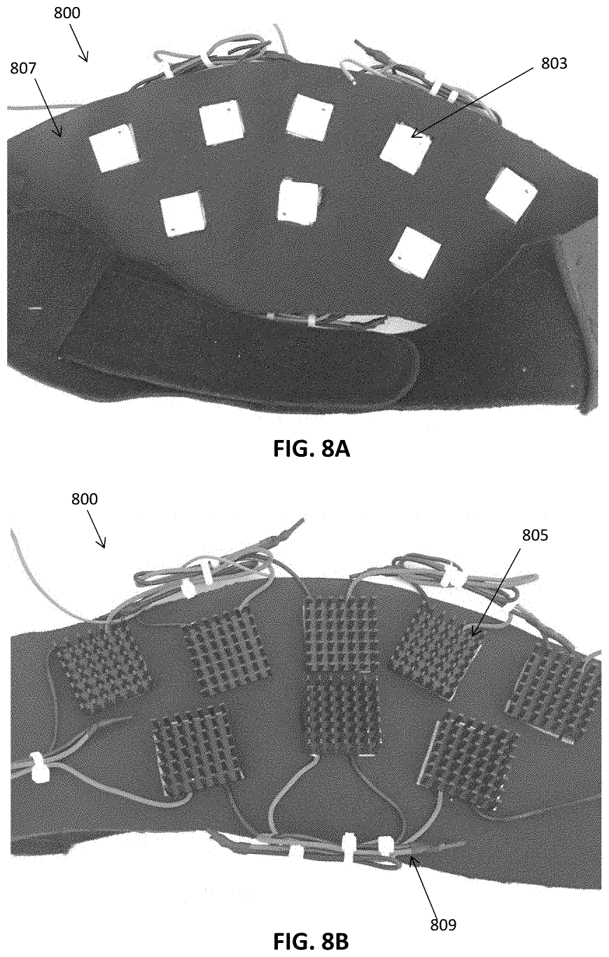

FIGS. 8A-8B illustrate an example of an apparatus as described herein. In some variations this apparatus may be used for, for example, enhancing sleep onset and/or maintaining sleep and/or treating a sleep-related (e.g., neurological) disorder, improving mood and/or increasing relaxation. In this example, a prototype forehead worn apparatus was built having a flexible, wearable substrate 807 that is thermally insulative, and through which a plurality of thermoelectric temperature regulators 803 are attached. The cooling side 803 of the thermoelectric temperature regulators are on the skin-facing side, while the warming side 805 are on the opposite side of the apparatus, as shown in FIG. 8B, configured to be worn facing away from the forehead.

In FIG. 8A, eight 15 mm by 15 mm thermoelectric temperature regulators are shown attached at discrete location for contacting the surface of a subject's skin. Each thermoelectric temperature regulators is connected (in the prototype wires 809 are visible, though they may be integrated into the fabric and/or covered. The thermoelectric temperature regulators may provide up to 8 W of cooling (e.g., between 2-8 W, between 3-7 W, between 4-8 W, etc.) using, for example, a power source such as a battery (e.g., Li battery, such as a 0.75″ by 2.5″ inch battery) that may be rechargeable.

In FIG. 8A, the device was tested and shown to be able to cool a subject's forehead over three hours of near-continuous use. The apparatus controller (not visible in FIGS. 8A-8B) may be included in the apparatus and may regulate the power to the apparatus. In FIG. 8B, the opposite side of the apparatus, which is worn so that it faces outward, shows the exposed backs of the thermoelectric temperature regulators to which a heat sink 805 has been attached. In practice, the battery or batteries (not shown in FIGS. 8A-8B) may be mounted on the wearable apparatus along with the control circuitry. The entire apparatus may be worn against the skin of the forehead, as shown in FIG. 5, above. The example devices shown above include a strap 813 holding the conformable temperature-regulated cooling surface of the thermoelectric temperature regulators against the skin; however, in some variations no strap is needed. The device may be adhesive held to the forehead surface, or other attachments may be used.

In any of the methods and apparatuses described herein, the apparatus may adjust the treatment (e.g., the temperature and/or timing) according to the subject's sleep cycle. Alternatively or additionally, any of the methods and apparatuses described herein may adjust the treatment based on the state of the subject's autonomic nervous system (e.g., sympathetic to parasympathetic ratio) and/or based on the response of the subject's parasympathetic and/or sympathetic nervous system. For example, these methods and apparatuses may include one or more sensors for measuring an indicator of the subject's autonomic nervous system response; this sensor data may be interpreted by the controller/processor, and may be used to adjust one or more of the temperature and/or timing of the therapy applied by the applicator to the subject's head (e.g., feedback).

Any appropriate sensed data for determining sleep stage (awake, NREM, REM, etc.) and/or determining the state of the autonomic nervous system (e.g., parasympathetic/sympathetic) may be used. For example, ECG, EEG, heart rate, heart rate variability, blood pressure, galvanic skin response, and/or any other indicator known to monitor autonomic function, an in particular parasympathetic function may be used.

It may be particularly helpful, but not necessary, to use one or more sensors configured to detect when a subject wearing the apparatus is experiencing a diving reflex. As mentioned, the apparatus may use feedback to adjust the temperature of the device and/or to switch between an active cooling (e.g., in a first range of between 0° C. and 25° C., or other cooling range) and a standby temperature (e.g., of between 26° C. and 38° C., e.g., between 30° C. and 38° C., between 32° C. and 38° C., between 34° C. and 38° C., etc.).

For example, a diving reflex may be detected by detecting peripheral vasoconstriction, slowed pulse rate, redirection of blood to the vital organs to conserve oxygen, release of red blood cells stored in the spleen, and heart rhythm irregularities. One or more sensors that detect and/or characterize a subject's diving reflex may be used in any of the methods an apparatuses describe herein. For example, the diving reflex may typically cause a change in heart rate of between 5-35% (e.g., 10-25%) within a few minutes (e.g., within 5 minutes, within 4 minutes, within 3 minutes, within 2 minutes, within 60 seconds, within 55 seconds, within 50 seconds, within 45 seconds, within 40 seconds, within 35 seconds, within 30 seconds, within 25 seconds, within 20 seconds, within 15 seconds, within 10 seconds, etc.). Thus, any of the apparatuses described herein may include a sensor configured to detect heat rate; this sensor(s) may be present on the applicator, or the sensor(s) may be separate from the applicator but in communication with the processor of the apparatus. For example, the subject may wear a wearable sensor that communicates with the apparatus. Sensors for detecting hear rate may include electrical (e.g., ECG) sensors, optical sensors (e.g., pulse oximetry sensors), vibration/motion sensors (e.g., accelerometers), etc. Alternatively or additionally, one or more sensors for detecting peripheral vasoconstriction may be used, and may be integrated into the apparatus or may communicate with the apparatus (e.g., pulse oximetry from one or preferably more locations, such as the hand/arm/finger and forehead). Changes in red blood cell levels may also be noninvasively detected and used to detect the presence and/or magnitude of a diving reflex.

One or more sensors may be included as part of the apparatus, including as part of the forehead applicator, as shown, or they may be separate from the applicator. As mentioned, these sensors may be for detecting one or more of: heart rate, heart rate variability, blood pressure, electroencephalogram, electrocardiogram, galvanic skin response, etc. The sensors may provide data to the processor/controller of the apparatus, where this data may be interpreted to determine the parasympathetic response or status of the subject. For example, the apparatus may be configured to determine if the subject is experiencing a diving reflex, or how robust a diving reflex the subject is experiencing, and may adjust the timing and temperature accordingly.

For example, any of the methods and apparatuses described herein may be configured to adjust temperature and/or timing of the apparatus based on the EEG, HRV and/or other sleep monitoring techniques, by themselves or in conjunction with an indicator of the diving reflex. The apparatus may vary the temperature applied throughout the sleep period based on feedback signals including feedback reflecting the sleep state or stage (e.g., awake, NREM (stage 1, stage 2, stage 3), REM etc.) and the diving reflex. In some variation the apparatus may adjust the temperature of the applicator in order to achieve and maintain a diving reflex in the subject, as determined by one or more sensors.

In one example, the temperature of the applicator may be controlled based on the heart rate. For example, the processor may monitor the heart rate to identify a change from an initial heart rate to a drop of more than 10% (e.g., between 10-35%) from the initial heart rate within a predetermined time period (e.g., 5 minutes, 4 minute, 3 minutes, 2 minutes, 1 minute, etc.), which may indicate the diving reflex. In a subject that is not yet asleep, the applicator may be cooled to a temperature that is ramped down (e.g., from body temperature, e.g., 37° C., or room temperature) gradually until the diving reflex is detected. Upon detection of the diving reflex (using one or more indicator, such as HR, HRV, blood pressure, vasoconstriction, rise in red blood cells, etc.) the temperature may be held steady. This procedure may therefore allow the cooling temperature to be customized to each subject/subject and for an individual subject between sessions, as some subjects may respond to a much lower or higher temperature to the induction of the diving reflex.

Detection of the diving reflex may also or alternatively be used to start a timing for the application of the temperature regulation of the therapy. For example, the temperature of the apparatus may be held at or below the temperature at which a diving reflex response is determined for a predetermined maintenance time period (e.g., 10 minutes, 15 minute, 20 minutes, 25 minutes, 30 minutes, 35 minutes, etc.) and then increased to a second (e.g., standby) temperature for a second (e.g., standby) predetermined time period. The apparatus or method may then cycle one or more time through cooler temperatures (e.g., temperatures inducing a diving reflex, which may be the same as the first iteration or may be determined by monitoring the subject) and standby temperatures. In some variations, the temperature may be adjusted within a cycle, for example, in order to maintain the subject at the diving reflex.

In general, a processor/controller of the apparatus may receive the data from the one or more sensor(s) and may analyze and interpret the data. As mentioned above, the processor may be part of the apparatus or it may, in some variations, be separate (e.g., remote) from the apparatus, such as a smart phone processor to which the apparatus communicates.

Any of the apparatuses described herein may include a conformable body configured to be worn on the subject's forehead having a skin-facing surface, as described above. In some variations the conformable body may include a rigid or semi-rigid frame that includes one or more parts that may be hinged or otherwise connected to each other and may hold the one or more thermoelectric temperature regulator(s). As described above, in some variations the thermoelectric temperature regulators are connected directly to complaint material, which may be a woven or non-woven fabric material. The conformable body may also include a thermally conductive skin-facing surface (e.g., a surface that is configured to be worn against the wearer's forehead. The skin-contacting surface may have a high thermal conductivity. These materials may be polymers, particularly polymers including a high-thermal conductivity filler or dopant material (e.g. metallic, ceramic, etc.). Other high-thermal conductivity materials include metallic materials (meshes, foils, etc.).

Thus, the conformable body may be a flexible frame. One or more straps, headband, hood or cap may be included with the conformable body or as part of the conformable body. In some variations the conformable body is held within the headband, hood or cap. The headband, hood or cap may include a window or opening either forming the skin-contacting surface or exposing the skin-contacting surface. In some variations all or part of the headband is formed of a mesh material. Either or both the conformable body and the headband, hood or cap may include darts or cut-outs to aid in conforming to the subject's head. In some variations, the headband, hood or cap includes extension tabs. The extension tabs may support the conformable body.

Any of these apparatuses may include one or more support layers for supporting the thermoelectric temperature regulator(s). For example, as mentioned above, the support layer may be a frame. Alternatively or additionally, the support layer may be a layer of fabric or the like.

The apparatuses described herein may be at least partially surrounded, including surrounded on the periphery and/or lateral sides, such as the sides perpendicular to the skin-facing surface with a thermal damping and/or vibration damping material(s). For example, the one or more thermoelectric temperature regulators may be at least laterally surrounded by one or more thermal damping and/or vibration damping materials. The thermal damping material may have a low thermal conductivity, particularly as compared to the thermal conductivity of the skin-contacting surface, which is thermally conductive. In some a thermal damping and vibration damping foam may be arranged around the one or more thermoelectric regulators. The foam may separate the fans and heatsinks and may reduce the sound of the fans.

Any of the apparatuses described herein may be configured to direct airflow from the apparatus, e.g., from one or more cooling fans. For example, the apparatus may direct airflow from the apparatus in a direction approximately parallel to the skin-contacting surface, e.g., up (toward the crown of the wearer's head when the wearer is wearing the apparatus. In some variations the airflow is directed laterally (e.g., towards the wearer's ears/side of the head when the wearer is wearing the apparatus). In some variations, the airflow is directed down (e.g., towards the wearer's nose/chin when the wearer is wearing the apparatus. In some variations airflow is directed up but prevented from blowing down and/or laterally. In some variations the airflow is directed laterally but prevented from blowing up and/or down. In some variations the airflow is directed down, but is prevented from blowing up and/or laterally. In some variations the airflow is directed perpendicularly to the skin-contacting surface.

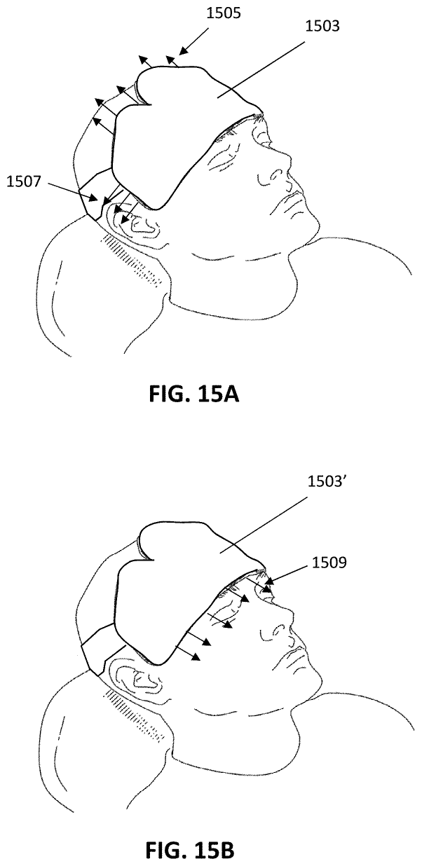

For example, FIGS. 15A and 15B illustrate two examples of apparatuses 1503, 1503′ configured to direct airflow in a target direction. In FIG. 15A, the apparatus 1503 is configured to include one or more outflow vents or channels that are configured to direct the (e.g., warmed) outflow of air 1505 from the thermoelectric temperature regulator in a direction that is up 1505, e.g., toward the crown of the wearer's head and laterally 1507 (e.g., towards the wearer's ears). Alternatively the airflow may be directed just laterally or just up. In some variations the airflow, which may be warmer air, may be directed so that some or all of it contacts the skin of the wearer; the contrast in temperature between the skin-contacting surface of the apparatus and the warmer air may enhance the effects of the apparatus as the difference between the perceived temperature and the applied temperature may be greater. Similarly, in some variations the region of the wearer's skin around the transfer region (e.g. at the periphery of the apparatus, may be warmed slightly (e.g., by between 0.5 and 4 degrees, between 0.5 and 3 degrees, between 0.5 and 2 degrees, etc.).

In FIG. 15B, the apparatus 1503′ is configured to emit the airflow (e.g., warmed air from cooling the thermoelectric temperature regulator, not visible in FIG. 15B) down 1509, e.g., towards the wearer's lower face. In any of these examples, some of the airflow may be directed away from the apparatus, e.g., perpendicular to the skin-facing (e.g., thermal transfer) surface of the apparatus.

Thus, as mentioned, the apparatus may include one or more vents or channels configured to direct airflow as described herein. Alternatively or additionally, the apparatuses described herein may draw air into the apparatus (e.g., to a cooling fan, etc.) via one or more vents or channels that are oriented in a particular direction. In some variations the inlet vents may be oriented in a direction that is opposite or normal to the direction of the outlet vents or channels discussed above.

In general, any of the apparatuses described herein may include a flexible outer layer to prevent material (e.g., hair, dust, etc.) from entering the apparatus, including being drawn into the fans. A flexible outer layer may be mesh or other material.

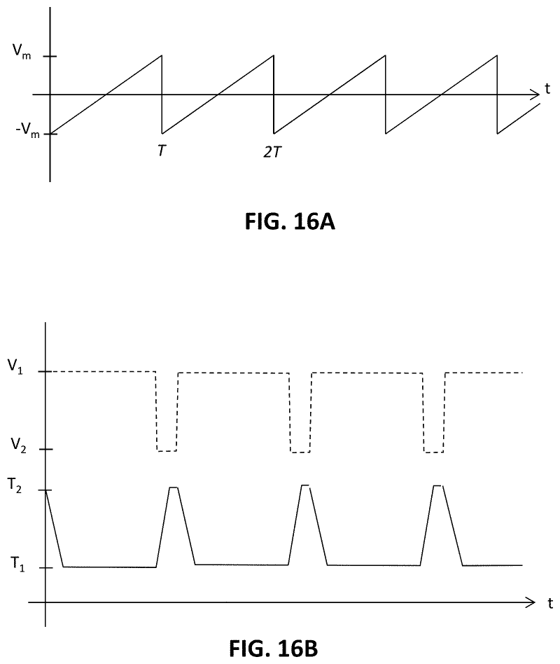

The apparatuses described herein may generally include a control unit comprising a controller, as discussed above. The controller may be configured to apply power using a control scheme to cool the thermoelectric temperature regulators to between 1° C.-30° C. The control scheme may be particularly well suited for triggering firing of thermal receptors in the skin. FIG. 16A is one illustration of a sawtooth control voltage that may be applied to one or more thermoelectric temperature regulators. The sawtooth control scheme shown may have any appropriate period T, or the period may be adjustable (e.g., thereby adjusting the frequency, e.g., between 0.1 and 10 kHz, between 1 and 1 kHz, between 10 and 300 Hz, etc.). In addition, the peak voltage may be any appropriate voltage and/or may be adjustable (e.g., between 0.001 and 50V, between 0.001 and 20 V, etc.). The applied energy may offset by a positive or negative DC offset voltage. In FIG. 16A, the sawtooth s centered on 0 V (and extends between +Vₘ and −Vₘ) in some variations the voltage goes from 0 to 2Vₘ, where 2Vₘ is the peak voltage. The set temperature may be adjusted based on the applied power (and therefore the applied sawtooth waveform).

FIG. 16B illustrates another example of a control scheme that may be used to regulate the power applied to the one or more thermoelectric temperature regulators. In FIG. 16B, the power is applied by the power control scheme to the one or more thermoelectric temperature regulators to cool the one or more thermally conductive skin-facing surfaces to a set temperature, e.g., between 1° C.-30° C., wherein the power control scheme varies the power so that the temperature of the one or more thermally conductive skin-facing surfaces deviates from the set temperature by between some predetermined amount (e.g., between about 1-5 degrees C.) over a predetermined period (e.g., of between about 7 seconds and 120 seconds). In FIG. 16B, the control scheme (dashed lines) includes a power on period in which the thermal transfer region is cooled by the TEC to a temperature (T₁) corresponding to the V₁ (set temperature voltage), then the held at that temperature for a predetermined amount of time (e.g., 7 seconds) then the power may be dropped to a lower level, e.g., V2, either instantaneously or by a ramp, and the temperature permitted to increase to the higher level (T₂), before the cycle is repeated, and the power is raised back to the V₁ level, decreasing the temperature (solid line) back to the T₁ temperature.

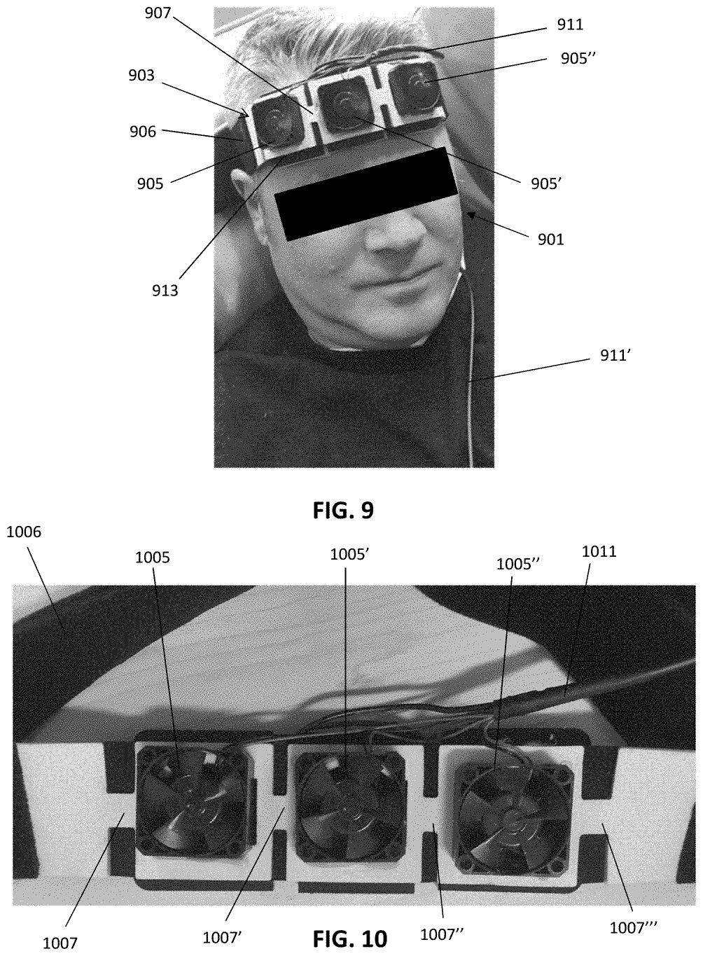

FIGS. 9-14 illustrate another example of an apparatus as described herein. In FIG. 9, the wearer 901 is shown wearing a prototype apparatus. In this example, the apparatus 903 includes three thermoelectric temperature regulators 905, 905′, 905″ that are connected to frame portions that are flexibly connected 907. The back of the thermoelectric temperature regulators are shown , each including a cooling fan portion; the apparatus may include channels or fins (not shown) for directing inlet and outlet of airflow to/from the thermoelectric temperature regulators. In addition a cover may be included. The apparatus is worn on the forehead, as shown, and may include one or more straps 906.

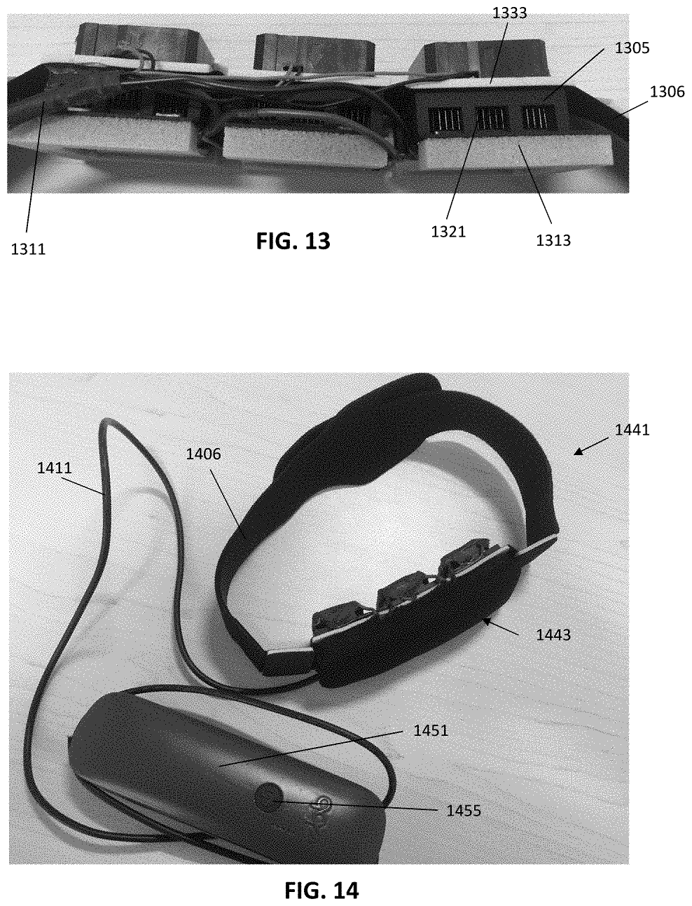

The apparatus may also include a cable or cord 911, 911′ that connects to the thermoelectric temperature regulator(s) and provides power and control. For example, as will be described in FIG. 14, the apparatus may include a separate housing enclosing control and/or power elements, connected to the forehead-worn portion by a cord or cable. This may allow easy control via one or more controls on the handheld unit (not shown) and/or powering from a battery or wall unit that is separately housed from the wearable portion. A thermal and/or vibration insulating or dampening material 913 may be included, as shown and described below in FIGS. 12-13.

FIG. 10 shows the prototype apparatus of FIG. 9 from the front, prior to being worn by a wearer. The three thermoelectric temperature regulators 1005, 1005′, 1005″ are coupled to a semi-rigid frame that is flexibly connected between each thermoelectric temperature regulators (e.g., through a narrow connecting region 1007, 1007′, 1007″, 1007′″. The flexible connector and/or frame may be formed of a flexible material, which may be part of the strap 1006 or connected to the strap, as shown. The cord 1011 is also shown extending from the head-worn portion of the apparatus.

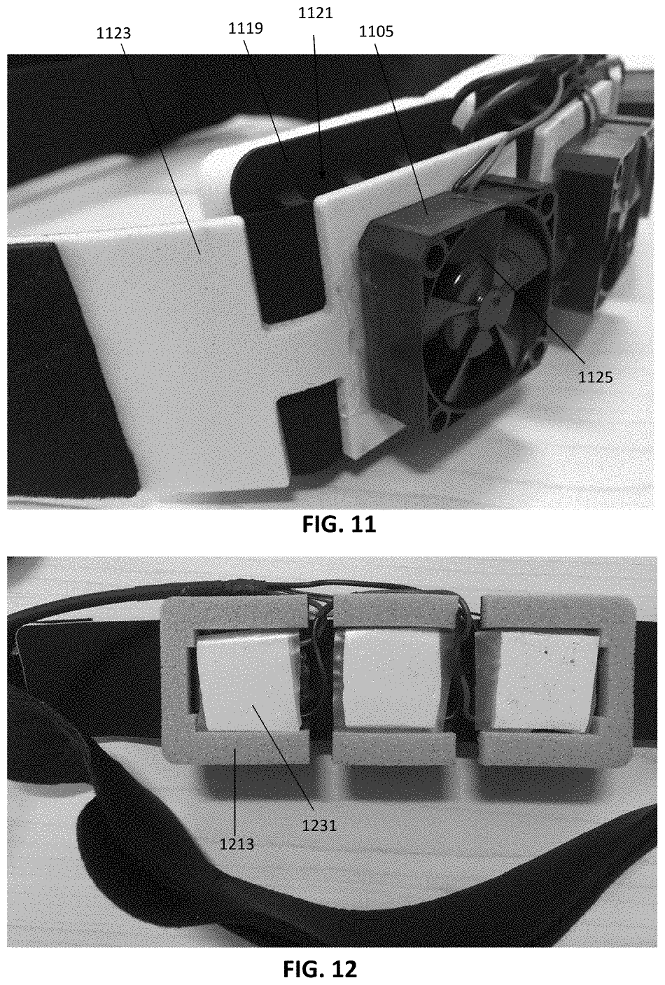

The flexible frame holding the thermoelectric temperature regulators 1105 is shown in the side view of FIG. 11. The frame 1119 includes a rigid polymeric portion that is attached to a flexible base portion 1123. The frame also include vents 1121 for outflow of air from the cooling fans 1125.

FIG. 12 shows a back view of the skin-contacting surface of the prototype apparatus of FIGS. 9-11. In FIG. 12, the prototype includes a foam material 1213 that at least partially laterally surrounds each of the bases of the thermoelectric temperature regulators (e.g., around or adjacent to the skin-contracting surfaces 1231). As described above, this foam material may be vibration-damping (e.g., reducing or eliminating the vibrations on or from the thermoelectric temperature regulators). The foam material may also be thermally insulating (e.g., thermally damping). In addition, the foam material may be compliant, increasing comfort for the wearer. FIG. 13 shows a top side view of the apparatus. The foam material 1313 laterally surrounds at least the outward sides of the base of the thermoelectric temperature regulators 1305 and the airflow path for air leaving the thermoelectric temperature regulator includes channels or fins 1321 directing the air up, towards the crown of the wearer's head. Each thermoelectric temperature regulators is mounted to a flexible frame 1333, which is also mounted or connected to the headband 1306. A cord 1311 connects the head-worn portion to a handheld controller, shown in FIG. 14.

In FIG. 14, the head worn portion 1441 connects via a cord 1411 to a housing 1451. The housing 1451 is configured as a hand-held housing that include a control (e.g., button 1455) and may enclose the controller (e.g., control circuitry, not visible in FIG. 14). The housing may also enclose a power source, such as a battery, and/or power control circuitry. In some variations the housing may connect to a power supply, such as a wall power source (e.g., outlet or power converter for converting wall power for use). In FIG. 14, the head-worn portion 1441 includes the strap or headpiece 1406, and this prototype apparatus is also shown with a cover 1443 covering the side of the apparatus opposite from the skin-contacting surface. This cover may be thermally conductive, as described above. A second or alternative cover may be used to cover the opposite side, e.g., the fans of the thermoelectric temperature regulators.

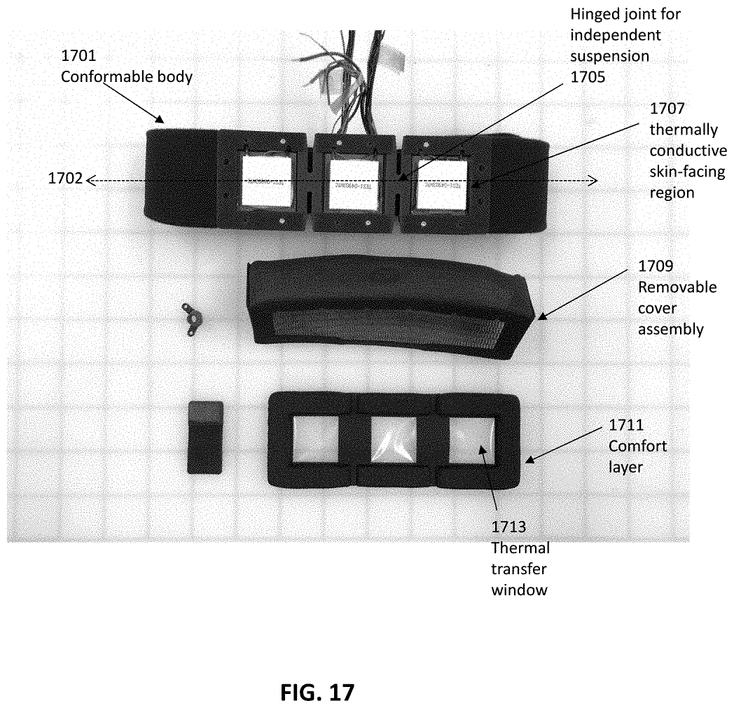

FIGS. 17-22 illustrate another example of an apparatus as described herein. In FIG. 17, the apparatus includes a conformable body 1701 that is configured to be worn on a subject's forehead. The conformable body has a plurality of thermally conductive skin-facing surfaces 1707 that are hinged together via a pivoting joint 1705. The pivoting joint in this example is formed by cutting the flexible material forming this skin-facing surface to form a notched or narrowed region that allows the thermally conductive skin surface to rotate around the long axis 1702 of the conformable body relative to each other. The long axis may curve or bend as the thermally conductive skin-facing surfaces bend. In some variations the thermally conductive skin-facing surfaces may be somewhat rigid, but are connected through this pivoting connection, allowing them to confirm to subject's having different head curvatures. Within each thermally conducive skin-facing surface, a region of thermal conductivity over the cooling/heating side of the attached TEC. As mentioned above, a gel pad or other thermal spreader may be included as part of the thermally conductive skin-facing surface.

FIG. 17 also show another example of a removable cover assembly 1709 that may be placed over the back side (not visible in FIG. 17) to cover the fan assemblies connected to each TEC and thermally conductive skin-facing surface. A comfort layer 1711 is also shown, which may fit over the skin-contacting surface and may include one or more thermally conductive windows 1713.

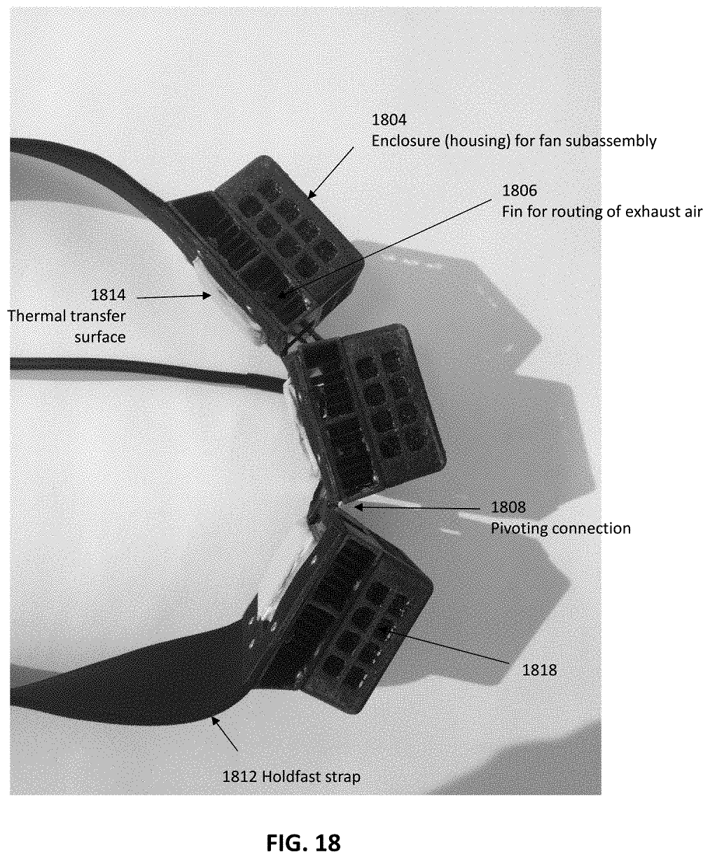

FIG. 18 is a side view of the apparatus of FIG. 17, without a comfort layer or removable cover assembly shown. In FIG. 18, the apparatus includes a thermal transfer surface 1814, and a plurality of thermoelectric temperature regulators (TECs) that are each in thermal communication with the one or more skin-facing surface. The thermally conductive skin-facing surfaces are each coupled to an adjacent thermally conductive skin-facing surface through a pivoting connection 1808 that permits each thermally conductive skin surfaces to rotate in the long axis relative to the adjacent skin-facing surface. A thermoelectric regulator assembly (including a fan sub-assembly 1818 and housing 1805) is connected to each thermal transfer surface and TEC (not visible in FIG. 18). Thus, each fan sub-assembly includes one or more cooling fans (not visible in FIG. 18) that is coupled to the conformable body through a mechanical damping member (not visible) such as a foam, within the housing or enclosure 1504. The fan sub-assemblies and TECs may be connected to a control unit (not visible in FIG. 18) and comprising a controller configured to apply power to the one or more thermoelectric temperature regulators to cool the one or more thermally conductive skin-facing surfaces, e.g., to between 1° C.-30° C.

The housing for the fan sub-assembly also includes inlets (not shown) and fan outlets 1806 that may direct the flow of air out of the device.

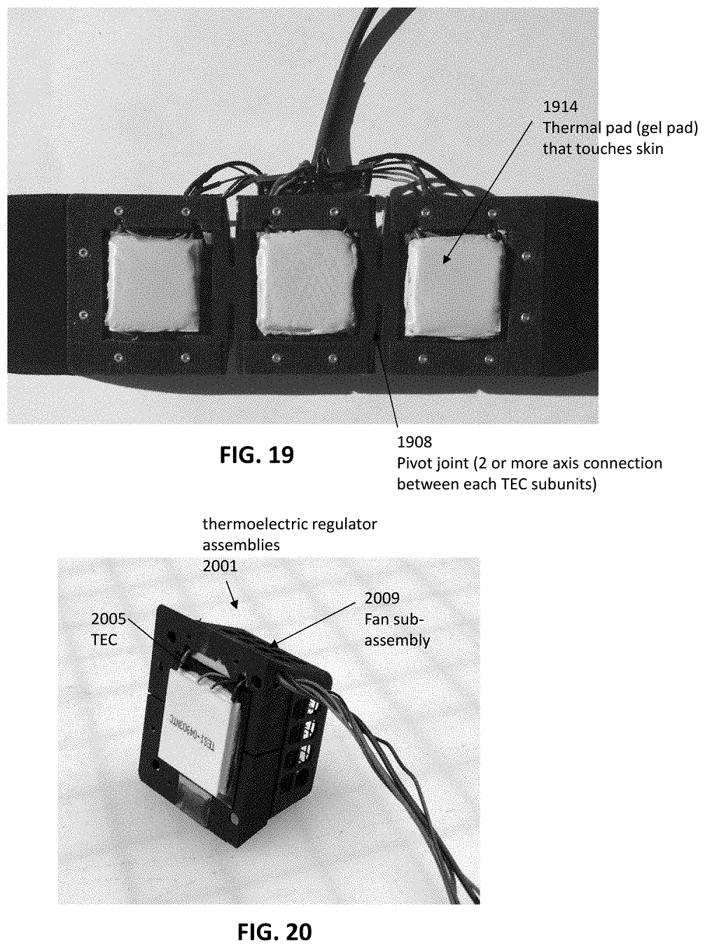

FIG. 19 shows a bottom view of the skin-facing surfaces of the apparatus of FIGS. 17-18. In this example, the pivot joint 1908 is visible between each skin-facing surface. A thermal pad (shown as a gel pad) 1914 may act as the thermal transfer region in communication with the TEC.

FIG. 20 illustrates one example of a thermoelectric regulator assembly 2001 that includes a fan sub-assembly 2009 and a TEC 2005. The fan sub-assembly may include a fan that is floating relative to the conformable body and housing, so that the fan is not rigidly connected to the conformable body or housing. For example, the fan may rest on a foam material that provides vibrational damping over the range of operating vibrations generated by the fan (e.g., between 20 Hz and 200 Hz, e.g., between 50 Hz and 100 Hz, etc.).

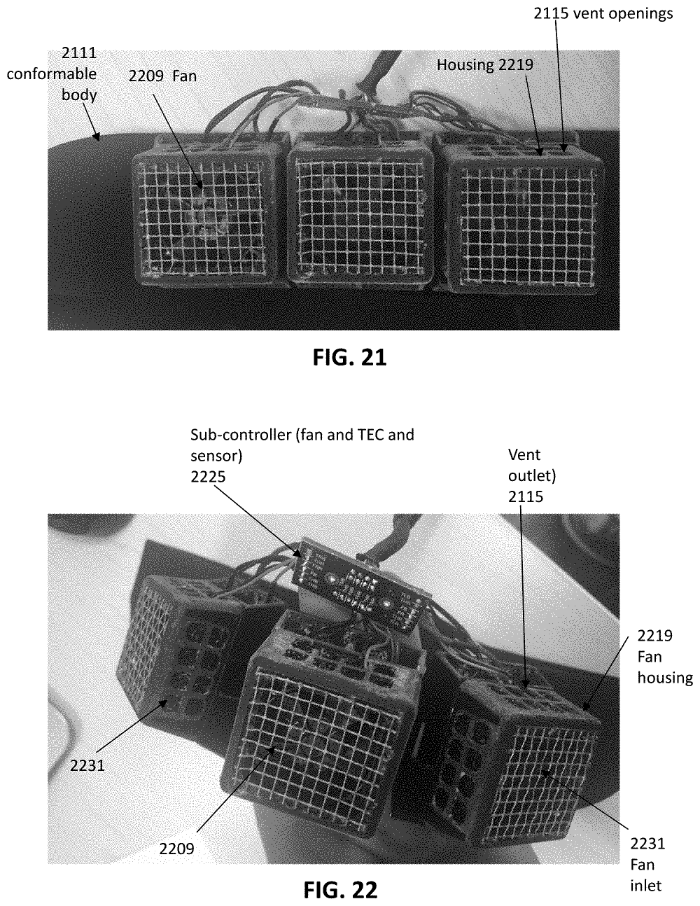

FIGS. 21 and 22 show top views of the apparatus, showing fan inlets 2231 on the surface of the fan housing 2219. An ‘unmounted’ (e.g. floating) fan 2209 is visible through the inlet openings in both FIGS. 21 and 22. The housings for each fan sub-assembly include vent openings (outlets) 2115 on the sides. The fan sub-assemblies may be attached to the conformable body 2111, and the power and control circuitry for each fan and TEC may be routed through a sub-controller 2225 and/or to a controller (not visible). In FIGS. 21 and 22, the mechanical damping member comprises an open-cell foam 2231 that is visible though the outlet openings on the side of the housing.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An apparatus comprising:
a conformable body configured to be worn on the subject's forehead having one or more thermally conductive skin-facing surfaces;
one or more thermoelectric temperature regulators (TERs) in thermal communication with the one or more skin-facing surfaces;
one or more cooling fans coupled to the conformable body through a mechanical damping member; and
a controller configured to apply power to the one or more TERs to cool or heat the one or more thermally conductive skin-facing surfaces to between 1° C.-30° C.

2. The apparatus of claim 1, wherein the mechanical damping member has a loss factor of greater than 0.8.

3. The apparatus of claim 1, wherein the mechanical damping member is configured to dampen vibrations between 300 and 600 Hz.

4. The apparatus of claim 1, wherein the mechanical damping member comprises an open-cell foam.

5. The apparatus of claim 1, wherein the conformable body comprises a plurality of rigid sections arranged in a long axis and linked by flexible hinge regions that are adapted to allow the skin-facing surfaces to conform the subject's forehead.

6. The apparatus of claim 5, wherein each rigid section includes a thermoelectric temperature regulator of the one or more TERs.

7. The apparatus of claim 1, wherein the controller is configured to apply a power control scheme to the one or more TERs comprising:
(a) applying power to the one or more TERs to cool or heat the one or more thermally conductive skin-facing surfaces to a set temperature of between 1-30° C.,
(b) holding the set temperature for between 1 and 15 seconds,
(c) reducing or stopping power to the one or more TERs until a temperature of the thermally conductive skin-facing surface varies from the set temperature by between 1-5° C., and
repeating steps (a)-(c).

8. The apparatus of claim 1, further comprising a removable comfort layer assembly attached to the conformable body over the one or more thermally conductive surfaces.

9. The apparatus of claim 8, wherein the comfort layer comprises a plurality of windows of thermally conductive material configured to interface over the thermally-conductive skin-facing surfaces.

10. The apparatus of claim 9, wherein the windows comprise polyurethane.

11. The apparatus of claim 1, further comprising a removable cover assembly configured to attach over the conformable body to cover the one or more cooling fans.

12. The apparatus of claim 1, further comprising an enclosure over each of the one or more TERs comprising one or more vents configured to direct airflow from the fans and parallel to the subject's skin and towards the crown of the subject's head or towards the subject's face but not laterally towards the subject's ears when the apparatus is worn on the subject's forehead.

13. The apparatus of claim 1, further comprising a headband or strap configured to secure the apparatus to the subject's head.

14. The apparatus of claim 1, further wherein the controller is enclosed in a controller housing connected by an elongate flexible cable to the one or more thermoelectric temperature regulators.

15. The apparatus of claim 14, further comprising a battery in the housing.

16. The apparatus of claim 1, further comprising one or more sensors configured to detect one or more of the subject's autonomic state and the subject's sleep/wake state, wherein the controller is configured to adjust the power to the one or more TERs based on input from the sensor.

17. A method of treating a subject to improve or enhance sleep, the method comprising:
applying, by a controller, a control scheme to a plurality of thermoelectric temperature regulators in communication with a skin-facing surface of a conformable body of an apparatus worn on a subject's forehead, wherein the control scheme repeatedly:
applies power to the thermoelectric temperature regulators to cool or heat the thermally conductive skin-facing surface in thermal communication with the thermoelectric temperature regulator to a set temperature of between 1-30° C.,
maintains the set temperature for a dwell time of between 1 and 15 seconds, and
reduces or stops power to the thermoelectric temperature regulators until a temperature of the thermally conductive skin-facing surface varies from the set temperature by between 1-5° C.

18. The method of claim 17, further comprising driving one or more cooling fans to cool the thermoelectric temperature regulators while damping vibration of the one or more cooling fans.

19. The method of claim 18, further comprising directing airflow from the one or more cooling fans parallel to the subject's skin and away from the subject's ears.

20. The method of claim 18, wherein damping vibrations comprises vibrationally isolating the one or more fans using a mechanical damping member that dampens vibrations between 300 and 600 Hz.

21. The method of claim 17, further comprising applying a strap of headband over the subject's head to secure the apparatus the apparatus to the subject's forehead.

22. The method of claim 17, further comprising operating the controller from a controller housing that is connected to the plurality of thermoelectric temperature regulators by an elongate, flexible cable.

23. The method of claim 17, wherein the power control scheme stops power to the thermoelectric temperature regulators until the temperature of the thermally conductive skin-facing surface varies from the set temperature by between 1-5° C.

24. The method of claim 17, wherein the power control scheme dynamically change the dwell time.

25. The method of claim 17, wherein the power control scheme decrease the dwell time as the apparatus is continuously operated.

26. The method of claim 17, further comprising detecting one or more of the subject's autonomic state and the subject's sleep/wake state, further wherein the controller is configured to adjust power to the thermoelectric temperature regulators based on input from the sensor.

27. The method of claim 17, further comprising detecting one or more of the subject's autonomic state and the subject's sleep/wake state and adjusting power to the thermoelectric temperature regulators based on the subject's autonomic state or the subject's sleep/wake state.

28. The method of claim 27, further comprising switching the control scheme to a standby mode based on one or both of the subject's autonomic state and the subject's sleep/wake state.

29. A method of treating a subject to improve or enhance sleep, the method comprising:
applying, by a controller, a control scheme to a plurality of thermoelectric temperature regulators in communication with a skin-facing surface of a conformable body of an apparatus worn on a subject's forehead, wherein the control scheme cyclically applies power to the thermoelectric temperature regulators to cool or heat the thermally conductive skin-facing surface in thermal communication with the thermoelectric temperature regulator so that a temperature of the thermally conductive skin-facing surface varies by between 1 and 5 degrees about a set temperature of between 1-30° C.; and
directing airflow from one or more cooling fans arranged to cool the plurality of thermoelectric temperature regulators so that the airflow is directed away from the subject's ears.

30. The method of claim 29, wherein directing airflow comprises directing the airflow so that it is parallel to the subject's skin.
